(19) United States
(12) Patent Application Publication
(10) Pub. No.: US 2007/0298489 A1
(43) Pub. Date: Dec. 27, 2007

(54) Novel human membrane proteins and polynucleotides encoding the same

(76) Inventors: Alejandro Abuin, The Woodlands, TX (US); Michael Burnett, Houston, TX (US); Gregory Donoho, Indianapolis, IN (US); Carl Johan Friddle, The Woodlands, TX (US); Glenn Friedrich, Houston, TX (US); Brenda Gerhardt, Spring, TX (US); Erin Hilbun, Denton, TX (US); Yi Hu, Spring, TX (US); Brian Mathur, Nashville, TN (US); Michael C. Nehls, Stockdorf (DE); Boris Nepomnichy, Dickinson, TX (US); Arthur T. Sands, The Woodlands, TX (US); John Scoville, Pearland, TX (US); C. Alexander Turner JR., The Woodlands, TX (US); D. Wade Walke, Spring, TX (US); Xiaoming Wang, Downers Grove, IL (US); Frank Wattler, Munich (DE); Nathaniel L. Wilganowski, Houston, TX (US); Brian Zambrowicz, The Woodlands, TX (US)

Correspondence Address: LEXICON PHARMACEUTICALS, INC., 8800 TECHNOLOGY FOREST PLACE, THE WOODLANDS, TX, 77381-1160, US

(21) Appl. No.: 11/244,415

(22) Filed: Oct. 5, 2005

Related U.S. Application Data
(63) Continuation-in-part of application No. 10/926,793, filed on Aug. 26, 2004, now abandoned.
Application No. 10/926,793 is a continuation of application No. 10/307,545, filed on Nov. 27, 2002, now abandoned.
Application No. 10/926,793 is a continuation of application No. 10/305,786, filed on Nov. 26, 2002, now abandoned.
Application No. 10/307,545 is a continuation of application No. 09/658,284, filed on Sep. 8, 2000, now abandoned.
Application No. 10/305,786 is a continuation of application No. 09/658,284, filed on Sep. 8, 2000, now abandoned.
(63) Continuation-in-part of application No. 09/658,283, filed on Sep. 8, 2000, now abandoned.
(63) Continuation-in-part of application No. 09/733,387, filed on Dec. 7, 2000, now abandoned.
(63) Continuation-in-part of application No. 11/095,093, filed on Mar. 31, 2005.
Application No. 11/095,093 is a continuation of application No. 09/755,017, filed on Jan. 5, 2001, now abandoned.
(63) Continuation-in-part of application No. 10/870,450, filed on Jun. 16, 2004, now abandoned.
Application No. 10/870,450 is a continuation of application No. 09/775,181, filed on Feb. 1, 2001, now abandoned.
(63) Continuation-in-part of application No. 09/783,669, filed on Feb. 14, 2001, now abandoned.
(63) Continuation-in-part of application No. 10/885,493, filed on Jul. 6, 2004, now abandoned.
Application No. 10/885,493 is a continuation of application No. 09/819,946, filed on Mar. 28, 2001, now abandoned.
(63) Continuation-in-part of application No. 11/022,049, filed on Dec. 22, 2004, now abandoned.
Application No. 11/022,049 is a continuation of application No. 09/878,764, filed on Jun. 11, 2001, now abandoned.
(63) Continuation-in-part of application No. 11/025,585, filed on Dec. 29, 2004, now abandoned.
Application No. 11/025,585 is a continuation of application No. 09/899,513, filed on Jul. 5, 2001, now abandoned.
(63) Continuation-in-part of application No. 09/916,122, filed on Jul. 26, 2001, now abandoned.
(63) Continuation-in-part of application No. 10/894,354, filed on Jul. 19, 2004, now abandoned.
Application No. 10/894,354 is a continuation of application No. 09/934,451, filed on Aug. 21, 2001, now abandoned.
(63) Continuation-in-part of application No. 09/975,308, filed on Oct. 11, 2001, now abandoned.
(63) Continuation-in-part of application No. 10/036,328, filed on Oct. 29, 2001, now abandoned.
(63) Continuation-in-part of application No. 10/008,574, filed on Oct. 26, 2001, now abandoned.
(63) Continuation-in-part of application No. 11/028,510, filed on Jan. 3, 2005, now abandoned.
Application No. 11/028,510 is a continuation of application No. 10/020,095, filed on Dec. 14, 2001, now abandoned.
(63) Continuation-in-part of application No. 10/990,309, filed on Nov. 16, 2004, now abandoned.
Application No. 10/990,309 is a continuation of application No. 10/390,567, filed on Mar. 17, 2003, now abandoned.
Application No. 10/390,567 is a continuation of application No. 10/042,810, filed on Jan. 9, 2002, now Pat. No. 6,570,003.
(63) Continuation-in-part of application No. 10/158,221, filed on May 29, 2002, now abandoned.
(63) Continuation-in-part of application No. 10/391,074, filed on Mar. 17, 2003, now abandoned.
Application No. 10/391,074 is a continuation of application No. 09/477,620, filed on Jan. 4, 2000, now abandoned.
(63) Continuation-in-part of application No. 10/443,530, filed on May 22, 2003, now abandoned.
Application No. 10/443,530 is a continuation of application No. 09/689,597, filed on Oct. 13, 2000, now abandoned.
(63) Continuation-in-part of application No. 10/394,962, filed on Mar. 21, 2003, now abandoned.
Application No. 10/394,962 is a continuation of application No. 09/843,164, filed on Apr. 27, 2001, now abandoned.
(63) Continuation-in-part of application No. 10/435,341, filed on May 9, 2003, now abandoned.
Application No. 10/435,341 is a continuation of application No. 09/852,909, filed on May 11, 2001, now abandoned.
(60) Provisional application No. 60/152,747, filed on Sep. 8, 1999.
(60) Provisional application No. 60/152,747, filed on Sep. 8, 1999.
(60) Provisional application No. 60/165,510, filed on Nov. 15, 1999.
(60) Provisional application No. 60/153,366, filed on Sep. 10, 1999.
(60) Provisional application No. 60/169,427, filed on Dec. 7, 1999.
(60) Provisional application No. 60/175,764, filed on Jan. 12, 2000.
(60) Provisional application No. 60/180,414, filed on Feb. 4, 2000.
(60) Provisional application No. 60/183,581, filed on Feb. 18, 2000.
(60) Provisional application No. 60/192,978, filed on Mar. 28, 2000.
(60) Provisional application No. 60/210,608, filed on Jun. 9, 2000.
(60) Provisional application No. 60/217,600, filed on Jul. 11, 2000.
(60) Provisional application No. 60/221,012, filed on Jul. 27, 2000.
(60) Provisional application No. 60/227,105, filed on Aug. 22, 2000.
(60) Provisional application No. 60/239,592, filed on Oct. 11, 2000.
(60) Provisional application No. 60/244,285, filed on Oct. 30, 2000.
(60) Provisional application No. 60/244,291, filed on Oct. 30, 2000.
(60) Provisional application No. 60/243,948, filed on Oct. 27, 2000.
(60) Provisional application No. 60/255,566, filed on Dec. 14, 2000.
(60) Provisional application No. 60/261,624, filed on Jan. 9, 2001.
(60) Provisional application No. 60/295,089, filed on May 29, 2001.
(60) Provisional application No. 60/114,666, filed on Jan. 4, 1999.
(60) Provisional application No. 60/115,828, filed on Jan. 14, 1999.
(60) Provisional application No. 60/159,150, filed on Oct. 13, 1999.
(60) Provisional application No. 60/199,950, filed on Apr. 27, 2000.
(60) Provisional application No. 60/203,875, filed on May 12, 2000.
(60) Provisional application No. 60/207,932, filed on May 30, 2000.

(51) Int. Cl. C12N 15/00; C07H 21/04; C07K 14/00

(52) U.S. Cl. 435/320.1; 530/350; 536/23.5

(57) ABSTRACT

Novel human polynucleotide and polypeptide sequences are disclosed that can be used in therapeutic, diagnostic, and pharmacogenomic applications.

### 1.0 CROSS-REFERENCE TO RELATED APPLICATIONS

The present application is a continuation-in-part of: co-pending U.S. application Ser. No. 10/926,793, filed on Aug. 26, 2004, which is a continuation of U.S. application Ser. Nos. 10/307,545, filed on Nov. 27, 2002, abandoned, and 10/305,786, filed on Nov. 26, 2002, abandoned, both of which are continuations of U.S. application Ser. No. 09/658,284, filed on Sep. 8, 2000, abandoned, which claims the benefit of U.S. Provisional Application No. 60/152,747, filed on Sep. 8, 1999; co-pending U.S. application Ser. No. 09/658,283, filed on Sep. 8, 2000, which claims the benefit of U.S. Provisional Application Nos. 60/165,510, filed on Nov. 15, 1999, and 60/153,366, filed on Sep. 10, 1999; co-pending U.S. application Ser. No. 09/733,387, filed on Dec. 7, 2000, which claims the benefit of U.S. Provisional Application No. 60/169,427, filed on Dec. 7, 1999; co-pending U.S. application Ser. No. 11/095,093, filed on Mar. 31, 2005, which is a continuation of U.S. application Ser. No. 09/755,017, filed on Jan. 5, 2001, abandoned, which claims the benefit of U.S. Provisional Application No. 60/175,764, filed on Jan. 12, 2000; co-pending U.S. application ser. No. 10/870,450, filed on Jun. 16, 2004, which is a continuation of U.S. application Ser. No. 09/775,181, filed on Feb. 1, 2001, abandoned, which claims the benefit of U.S. Provisional Application No. 60/180,414, filed on Feb. 4, 2000; co-pending U.S. application Ser. No. 09/783,669, filed on Feb. 14, 2001, which claims the benefit of U.S. Provisional Application No. 60/183,581, filed on Feb. 18, 2000; co-pending U.S. application Ser. No. 10/885,493, filed on Jul. 6, 2004, which is a continuation of U.S. application Ser. No. 09/819,946, filed on Mar. 28, 2001, abandoned, which claims the benefit of U.S. Provisional Application No. 60/192,978, filed on Mar. 28, 2000; co-pending U.S. application Ser. No. 11/022,049, filed on Dec. 22, 2004, which is a continuation of U.S. application Ser. No. 09/878,764, filed on Jun. 11, 2001, abandoned, which claims the benefit of U.S. Provisional Application No. 60/210,608, filed on Jun. 9, 2000; co-pending U.S. application Ser. No. 11/025,585, filed on Dec. 29, 2004, which is a continuation of U.S. application Ser. No. 09/899,513, filed on Jul. 5, 2001, abandoned, which claims the benefit of U.S. Provisional Application No. 60/217,600, filed on Jul. 11, 2000; co-pending U.S. application Ser. No. 09/916,122, filed on Jul. 26, 2001, which claims the benefit of U.S. Provisional Application No. 60/221,012, filed on Jul. 27, 2000; co-pending U.S. application Ser. No. 10/894,354, filed on Jul. 19, 2004, which is a continuation of U.S. application Ser. No. 09/934,451, filed on Aug. 21, 2001, abandoned, which claims the benefit of U.S. Provisional Application No. 60/227,105, filed on Aug. 22, 2000; co-pending U.S. application Ser. No. 09/975,308, filed on Oct. 11, 2001, which claims the benefit of U.S. Provisional Application No. 60/239,592, filed on Oct. 11, 2000; co-pending U.S. application Ser. No. 10/036,328, filed on Oct. 29, 2001, which claims the benefit of U.S. Provisional Application No. 60/244,285, filed on Oct. 30, 2000; co-pending U.S. application Ser. No. 10/008,574, filed on Oct. 26, 2001, which claims the benefit of U.S. Provisional Application Nos. 60/244,291, filed on Oct. 30, 2000, and 60/243,948, filed on Oct. 27, 2000; co-pending U.S. application Ser. No. 11/028,510, filed on Jan. 3, 2005, which is a continuation of U.S. application Ser. No. 10/020,095, filed on Dec. 14, 2001, abandoned, which claims the benefit of U.S. Provisional Application No. 60/255,566, filed on Dec. 14, 2000; co-pending U.S. application Ser. No. 10/990,309, filed on Nov. 16, 2004, which is a continuation of U.S. application Ser. No. 10/390,567, filed on Mar. 17, 2003, abandoned, which is a continuation of U.S. application Ser. No. 10/042,810, filed on Jan. 9, 2002, which issued as U.S. Pat. No. 6,570,003 B1 on May 27, 2003, which claims the benefit of U.S. Provisional Application No. 60/261,624, filed on Jan. 9, 2001; co-pending U.S. application Ser. No. 10/158,221, filed on May 29, 2002, which claims the benefit of U.S. Provisional Application No. 60/295,089, filed on May 29, 2001; co-pending U.S. application Ser. No. 10/391,074, filed on Mar. 17, 2003, which is a continuation of U.S. application Ser. No. 09/477,620, filed on Jan. 4, 2000, abandoned, which claims the benefit of U.S. Provisional Application Nos. 60/114,666, filed on Jan. 4, 1999, and 60/115,828, filed Jan. 14, 1999; co-pending U.S. application Ser. No. 10/443,530, filed on May 22, 2003, which is a continuation of U.S. application Ser. No. 09/689,597, filed on Oct. 13, 2000, abandoned, which claims the benefit of U.S. Provisional Application No. 60/159,150, filed on Oct. 13, 1999; co-pending U.S. application Ser. No. 10/394,962, filed on Mar. 21, 2003, which is a continuation of U.S. application Ser. No. 09/843,164, filed on Apr. 26, 2001, abandoned, which claims the benefit of U.S. Provisional Application No. 60/199,950, filed on Apr. 27, 2000; and co-pending U.S. application Ser. No. 10/435,341, filed on May 9, 2003, which is a continuation of U.S. application Ser. No. 09/852,909, filed on May 11, 2001, abandoned, which claims the benefit of U.S. Provisional Application Nos. 60/203,875, filed on May 12, 2000, and 60/207,932, filed on May 30, 2000; each of which is herein incorporated by reference in its entirety.

### 2.0 CROSS-REFERENCE TO SEQUENCE LISTING SUBMITTED ON COMPACT DISC

The present application contains a Sequence Listing of SEQ ID NOS:1-257, in file “Seqlist.txt” (1,656,832 bytes), created on Oct. 5, 2005, submitted herewith on duplicate compact disc (Copy 1 and Copy 2), which is herein incorporated by reference in its entirety.

### 3.0 INTRODUCTION

The present invention relates to the discovery, identification, and characterization, of novel human polynucleotides that encode membrane proteins, membrane-associated proteins, secreted proteins, and receptors. The invention encompasses the described polynucleotides, host cell expression systems, the encoded proteins, fusion proteins, polypeptides and peptides, antibodies to the encoded proteins and peptides, and genetically engineered animals that lack the disclosed genes, or overexpress the disclosed genes, or antagonists and agonists of the proteins, and other compounds that modulate the expression or activity of the proteins encoded by the disclosed genes that can be used for diagnosis, drug screening, clinical trial monitoring, the treatment of physiological or behavioral disorders, and/or cosmetic or nutriceutical applications.

### 4.0 BACKGROUND OF THE INVENTION

In addition to providing the structural and mechanical scaffolding for cells and tissues, membrane proteins can also serve as recognition markers, mediate signal transduction, and can mediate or facilitate the passage of materials across the lipid bilayer. As such, membrane proteins are good drug targets, and soluble forms thereof can directly serve as therapeutics.

Alpha macroglobulins are large secreted glycoproteins present in vertebrate blood plasma that have been implicated in modulating protease activity, and a number of associated biological processes and anomalies.

Membrane receptor proteins can serve as integral components of cellular mechanisms for sensing their environment, and maintaining cellular homeostasis and function. Accordingly, membrane receptor proteins are often involved in transduction pathways that control cell physiology, chemical communication, and gene expression.

Sensory receptor proteins are typically membrane proteins that interact with ligands or stimuli and mediate signal transduction. As such, extracellular receptor proteins are good drug or chemical targets.

A particularly relevant class of membrane receptors are those typically characterized by the presence of 7 conserved transmembrane domains that are interconnected by nonconserved hydrophilic loops. Such, seven transmembrane receptors, or 7TM receptors, include a superfamily of receptors known as G protein-coupled receptors (GPCRs). GPCRs are typically involved in transduction pathways involving G proteins or PPG proteins. As such, the GPCR family includes many receptors that are known to serve as drug targets for therapeutic agents.

### 5.0 SUMMARY OF THE INVENTION

The present invention relates to the discovery, identification, and characterization of nucleotides that encode novel human membrane proteins, many of which are GPCRs, and the corresponding novel amino acid sequences. The GPCRs described for the first time herein are transmembrane proteins that span the cellular membrane and are involved in signal transduction after ligand binding. The described GPCRs have structural motifs found in the 7TM receptor family. Expression of the described membrane proteins can be detected in a variety of human cells.

The novel human sequences described herein encode membrane proteins of 371, 1250, 1221, 718, 1112, 1249, 1220, 717, 1111, 1250, 1221, 718, 1112, 541, 512, 8, 403, 1222, 1193, 690, 1084, 1221, 1192, 689, 1083, 1222, 1193, 690, 1084, 1192, 225, 508, 298, 359, 233, 162, 504, 294, 355, 229, 158, 521, 311, 372, 246, 175, 485, 275, 336, 210, 139, 549, 339, 400, 274, 203, 313, 1215, 599, 197, 270, 253, 202, 841, 763, 366, 234, 910, 598, 205, 1257, 1158, 324, 925, 300, 338, 298, 316, 307, 293, 357, 309, 310, 1210, 734, 1138, 662, 640, 578, 1445, 1428, 1248, 1278, 890, 898, 777, 775, 785, 783, 862, 860, 322, 552, 314, 314, 769, 296, 848, 481, 423, 560, 502, 241, 320, 994, 826, and 335 amino acids in length (see, respectively, SEQ ID NOS:2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 63, 65, 67, 69, 71, 73, 75, 77, 79, 81, 83, 85, 87, 89, 91, 93, 95, 97, 99, 101, 103, 105, 107, 109, 111, 113, 116, 119, 121, 124, 126, 128, 130, 133, 135, 137, 139, 142, 144, 146, 149, 151, 154, 157, 159, 162, 164, 166, 169, 172, 175, 177, 180, 182, 184, 186, 188, 191, 193, 196, 198, 201, 203, 206, 208, 210, 212, 214, 216, 218, 220, 223, 225, 227, 229, 233, 235, 237, 239, 241, 243, 245, 247, 249, 252, 254, and 257). SEQ ID NOS:61, 114, 117, 122, 131, 140, 147, 152, 155, 160, 167, 170, 173, 178, 189, 194, 199, 204, 221, 230, 231, 250, and 255 describe open reading frames (ORFs) and flanking regions. The described GPCRs have multiple transmembrane regions (of about 20-30 amino acids) characteristic of 7TM proteins, as well as several predicted cytoplasmic domains.

As evidenced by the alternative 5′ regions and splice junctions of the open reading frames present in SEQ ID NOS:3-61, the transcription of these sequences can apparently initiate from one of several different promoters present within the genome. Depending on the promoter that is used, these proteins can have one of several distinct amino acid sequences at or near the amino terminus. An additional feature of SEQ ID NOS:3-61 is that the 7TM region of the protein is typically present at the C-terminal portion of the protein (for example, beginning at about amino acid 710 of SEQ ID NO:4), and thus these proteins incorporate a large upstream open reading frame that is substantially similar to a variety of other proteins, such as bone morphogenic protein and several different proteases. Given that, in lower organisms, ORFs encoding receptor ligands can be linked to the ORF encoding the cognate receptor, it is possible that the presently described upstream ORF encodes a product (or cleavage product) that can serve as a receptor ligand in the body. Accordingly, an additional embodiment of the present invention includes soluble protein products or ligands that are encoded by at least a portion of SEQ ID NOS:3-61.

The invention encompasses the nucleotides presented in the Sequence Listing, expression vectors that have been engineered to incorporate one or more of the nucleotides presented in the Sequence Listing, host cells expressing such nucleotides, the expression products of such nucleotides, and: (a) nucleotides that encode mammalian homologs of the described membrane proteins, including the specifically described human membrane proteins, and the human membrane protein gene products; (b) nucleotides that encode one or more portions of the described membrane proteins that correspond to functional domains, and the polypeptide products specified by such nucleotide sequences, including, but not limited to, the novel regions of the described extracellular domain(s) (ECD), transmembrane domain(s) (TM), and cytoplasmic domain(s) (CD) first disclosed herein; (c) isolated nucleotides that encode mutants, engineered or naturally occurring, of the described membrane proteins, in which all or a part of at least one of the domains is deleted or altered, and the polypeptide products specified by such nucleotide sequences, including, but not limited to, soluble receptors, in which all or a portion of a TM is deleted (in the case of the described 7TMs, a soluble product can be generated by engineering a protein to include only the region upstream from the first TM, such that all downstream TMs are deleted), and nonfunctional receptors, in which all or a portion of one or more of the CD(s) is deleted; (d) nucleotides that encode fusion proteins containing all or a portion of the coding region from one or more of the described membrane proteins, or one of its domains (e.g., an extracellular domain), fused to another peptide or polypeptide; and (e) therapeutic or diagnostic derivatives of the described polynucleotides, such as oligonucleotides, antisense polynucleotides, ribozymes, double stranded RNA (dsRNA), or gene therapy constructs, comprising one or more of the sequences first disclosed in the Sequence Listing.

The invention also encompasses agonists and antagonists of the described membrane proteins (including small molecules, large molecules, mutant forms of the described membrane proteins, or portions thereof, that compete with the native membrane protein, and antibodies), nucleotide sequences that can be used to inhibit (e.g., antisense and ribozyme molecules, and gene or regulatory sequence replacement constructs) or enhance (e.g., expression constructs that place the described sequence under the control of a strong promoter system) expression of the described membrane proteins, and transgenic animals that express one or more of the described membrane protein transgenes, or “knock-outs” that do not express a functional version of the described membrane proteins.

Additionally contemplated are “knock-out” embryonic stem (ES) cells that have been engineered using conventional methods (see, e.g., U.S. Pat. No. 6,924,146 B1). When the unique membrane protein sequences described in SEQ ID NOS:1-257 are “knocked-out” they provide a method of identifying phenotypic expression of the particular gene, as well as a method of assigning function to previously unknown genes. In addition, animals in which a homolog of the unique membrane protein sequences described in SEQ ID NOS:1-257 are “knocked-out” provide a unique source in which to elicit antibodies to homologous and orthologous proteins, which would have been previously viewed by the immune system as “self” and therefore would have failed to elicit significant antibody responses. Accordingly, an additional aspect of the present invention includes knockout cells and animals having genetically engineered mutations in the sequences encoding the presently described membrane proteins. Knock-out mice can be produced in several ways, one of which involves the use of mouse ES cell lines that contain gene trap mutations in a murine homolog of at least one of the described human membrane proteins. To these ends, gene trapped knockout ES cells have been generated in murine homologs of the described membrane proteins.

Additionally, the unique membrane protein sequences described in SEQ ID NOS:1-257 are useful for the identification of protein coding sequences, and mapping unique genes to one or more particular chromosome. These sequences identify actual, biologically relevant, exon splice junctions, as opposed to those that might have been predicted bioinformatically from genomic sequence alone. The sequences of the present invention are also useful as additional DNA markers for restriction fragment length polymorphism (RFLP) analysis, and in forensic biology.

Further, the present invention also relates to methods of using the described membrane protein nucleotide sequences and/or gene products for the identification of compounds that modulate, i.e., act as agonists or antagonists of, gene expression and/or gene product activity of the described membrane proteins. Such compounds can be used as therapeutic agents for the treatment of various symptomatic representations of biological disorders or imbalances.

### 6.0 BRIEF DESCRIPTION OF THE FIGURES

No Figures are required in the present invention.

### 7.0 DETAILED DESCRIPTION OF THE INVENTION

The human polynucleotide sequences described for the first time herein encode novel membrane proteins that are expressed in, inter alia, human cell lines, and: human hypothalamus, fetal brain, brain, and cerebellum cells, with less but detectable expression in human heart, esophagus, fetal liver, and colon cells (SEQ ID NOS:1-2); human placenta, lung, kidney, liver, pancreas, spinal cord, spleen, thymus, lymph node, adrenal gland, stomach, salivary gland, mammary gland, thyroid, heart, brain, testis, kidney, adipose, esophagus, rectum, pericardium, trachea, fetal liver, prostate, small intestine, and colon cells (SEQ ID NOS:3-61); human spleen, bone marrow, and adipose cells (SEQ ID NOS:62-114); human brain, cerebellum, spinal cord, thymus, spleen, bone marrow, liver, placenta, prostate, thyroid, testis, adrenal gland, stomach, small intestine, colon, esophagus, bladder, rectum, pericardium, and fetal lung cells (SEQ ID NOS:115-117); human fetal brain, brain, cerebellum, hypothalamus, and testis cells,(SEQ ID NOS:118-122); human spinal cord, kidney, hypothalamus, and particularly adrenal gland and heart cells (SEQ ID NOS:123-131); human testis and kidney cells (SEQ ID NOS:132-140); human pituitary, spinal cord, lymph node, trachea, kidney, prostate, testis, thyroid, salivary gland, small intestine, heart, uterus, placenta, mammary gland, adipose, esophagus, bladder, cervix, fetal lung, and fetal kidney cells (SEQ ID NOS:141-144); human lymph node, brain, adrenal gland, bone marrow, pituitary, placenta, prostate, thymus, thyroid, and kidney cells (SEQ ID NOS:145-147); human fetal brain, cerebellum, spinal cord, spleen, kidney, fetal liver, liver, prostate, testis, adrenal gland, skeletal muscle, uterus, adipose, esophagus, cervix, rectum, and pericardium cells (SEQ ID NOS:148-152); human pituitary, spleen, lymph node, trachea, liver, testis, heart, uterus, adipose, esophagus, cervix, rectum, pericardium, fetal kidney, and fetal lung cells (SEQ ID NOS:153-155); human kidney, fetal kidney, and fetal lung cells (SEQ ID NOS:156-160); human spinal cord, spleen, lymph node, lung, testis, skeletal muscle, placenta, cervix, fetal lung, and 6 week embryo cells (SEQ ID NOS:161-167); human fetal brain, spinal cord, spleen, lymph node, bone marrow, testis, thyroid, adrenal gland, stomach, small intestine, colon, skeletal muscle, uterus, mammary gland, fetal kidney, and adenocarcinoma cells (SEQ ID NOS:168-170); human lymph node, lung, fetal liver, testis, small intestine, skeletal muscle, uterus, and bladder cells (SEQ ID NOS:171-173); human spinal cord, spleen, lung, skeletal muscle, placenta, fetal kidney, and fetal lung cells (SEQ ID NOS:174-178); human testis, fetal kidney, and aorta cells (SEQ ID NOS:179 and 180); human testis, small intestine, and uterus cells (SEQ ID NOS:181-189); human pituitary, testis, skeletal muscle, adipose, esophagus, cervix, pericardium, fetal kidney, and fetal lung cells (SEQ ID NOS:190-194); human fetal brain, brain, pituitary, spinal cord, thymus, spleen, lymph node, trachea, lung, kidney, fetal liver, liver, prostate, testis, thyroid, adrenal gland, pancreas, salivary gland, stomach, small intestine, colon, skeletal muscle, heart uterus, mammary gland, adipose, skin, bladder, cervix, pericardium, ovary, fetal kidney, fetal lung, gall bladder, tongue, embryo, sarcoma, carcinoma, and endothelial cells (SEQ ID NOS:195-199); human fetal brain, pituitary, spinal cord, thymus, spleen, lymph node, liver, prostate, testis, thyroid, adrenal gland, skeletal muscle, uterus, placenta, mammary gland, bladder, rectum, pericardium, ovary, fetal kidney, fetal lung, gall bladder, tongue, aorta, 6-, 9-, and 12- week embryos, adenocarcinoma, osteosarcoma, and endothelial cells (SEQ ID NOS:200-204); human fetal brain, pituitary, spinal cord, spleen, lymph node, testis, thyroid, adrenal gland, colon, skeletal muscle, uterus, placenta, and fetal kidney cells (SEQ ID NOS:205-221); human testis, mammary gland, and salivary gland cells (SEQ ID NOS:222 and 223); human brain, testis, kidney, and trachea cells (SEQ ID NOS:224 and 225); human pancreas, testis, adipose, esophagus, cervix, rectum, and pericardium cells (SEQ ID NOS:226-231); human placenta, bone marrow, trachea, testis, liver, and kidney cells (SEQ ID NOS:232-250); human placenta, bone marrow, trachea, testis, liver, and kidney cells (SEQ ID NOS:251-255); and human testis, heart, skeletal muscle, lung, adrenal gland, kidney, prostate, and lymph node cells (SEQ ID NOS:256 and 257).

Additionally, SEQ ID NOS:118-122 were used as nucleic acid hybridization probes to examine gene expression in normal mouse tissue sections. This analysis revealed expression of mRNA corresponding to the murine homolog of SEQ ID NOS:118-122 in numerous neurons throughout the mouse brain. Expression was particularly striking in a susbset of neurons in the internal granular layer of the cerebellum (presumably Golgi neurons), and in a collection of neurons that define the substantia nigra of the midbrain, associated with cells responsible for the synthesis of dopamine, the pars compacta, indicating potential involvement of SEQ ID NOS:118-122 in Parkinson's disease.

The described membrane protein sequences were compiled from: gene trapped human cells and a human placenta cDNA library (SEQ ID NOS:3-61); gene trapped human cells and cDNA clones isolated from human lymph node and bone marrow cDNA libraries (SEQ ID NOS:62-114); gene trapped human cells and a human placenta and thyroid cDNA libraries (SEQ ID NOS:115-117); gene trapped human cells and cDNA clones isolated from human brain and cerebellum cDNA libraries (SEQ ID NOS:118-122); gene trapped human cells and CDNA clones isolated from human kidney and testis cDNA libraries (SEQ ID NOS:132-140); human genomic sequences in conjunction with cDNAs isolated from a human kidney cDNA library (SEQ ID NOS:141-144); clustered human gene trapped sequences, genomic sequence, ESTs, and cDNAs from a human testis, skeletal muscle, mammary gland, and lymph node cDNA libraries (SEQ ID NOS:145-152); cDNAs present in adipose and testis cDNA libraries (SEQ ID NOS:153-155); human genomic sequences in conjunction with cDNAs generated from a human kidney mRNAs (SEQ ID NOS:156-160); human genomic sequences in conjunction with cDNAs generated from mRNAs from human skeletal muscle, testis, and spleen cells (SEQ ID NOS:161-180); human genomic sequences in conjunction with cDNAs generated from mRNAs from human testis, small intestine, and uterus (SEQ ID NOS:181-189); human genomic sequences in conjunction with cDNAs generated from mRNAs from human testis, pituitary, and adipose (SEQ ID NOS:190-194); aligning cDNAs from umbilical vein, lung, adrenal gland, mammary gland, prostate, thyroid, fetus, kidney, testis, and placenta mRNAs, and human genomic DNA sequence (SEQ ID NOS:195-199); human genomic sequences (GenBank Accession Number AC068148), in conjunction with cDNAs generated from mRNAs from human fetal brain, lung, and testis cells (SEQ ID NOS:200-204); from clustered genomic sequence, ESTs, and cDNAs produced using human placenta, pituitary gland, colon, testis, lymph node, skeletal muscle, fetus, thyroid, uterus, bone marrow, and adrenal gland mRNAs (SEQ ID NOS:205-221); sequences from gene trapped human cells and cDNA clones isolated from a human placenta cDNA library (SEQ ID NOS:226-231); sequences from gene trapped sequences and cDNA clones isolated from human trachea, liver, and testis cDNA libraries (SEQ ID NOS:232-250); human genomic sequences in conjunction with cDNAs isolated from human kidney and lymph node cDNA libraries (SEQ ID NOS:251-255); and cDNAs present in a skeletal muscle cDNA library (SEQ ID NOS:256 and 257). The mRNAs and/or cDNA libraries used were purchased from Clontech (Palo Alto, Calif.) and/or Edge Biosystems (Gaithersburg, Md.).

Many of the described membrane proteins are transmembrane proteins of the 7TM family of receptors. As with other GPCRs, signal transduction is triggered when a ligand binds to the receptor. Interfering with the binding of the natural ligand, or neutralizing or removing the ligand, or interference with its binding to a GPCR will effect GPCR-mediated signal transduction. Because of their biological significance, 7TM, and particularly GPCR, proteins have been subjected to intense scientific and commercial scrutiny (see, e.g., U.S. Pat. Nos. 5,942,416 and 5,891,720, for applications, uses, and assays involving GPCRs).

In addition, the presently described membrane proteins share significant homology with: neuropeptide hormone receptors, including the vasopressin receptor and its evolutionary precursor, the vasotocin receptor, the oxytocin receptor of mammals, the marsupial homologue the mesotocin receptor, and the isotocin receptor of fish (SEQ ID NOS:1 and 2); metabotropic amino acid, or glutamate, receptors, which have been implicated in neurodegeneration, seizures, schizophrenia, and other neural or behavioral disorders, and thus have been the subject of considerable study, as evidenced by U.S. Pat. Nos. 5,869,609, 5,912,122, 5,981,195, and 6,001,581, and U.S. Provisional Application Ser. No. 60/085,973 (SEQ ID NOS:118-122); mammalian taste and pheromone receptors, calcium sensing receptors, and peptide hormone receptors (SEQ ID NOS:132-140); a domain of latrophilin (latrotoxin receptor) and peptide hormone receptors (SEQ ID NOS:141-144); mammalian membrane ligand (particularly myrosinase) or lectin binding proteins (SEQ ID NOS:145-147); mammalian transporter proteins, particularly those associated with multi-drug resistance (SEQ ID NOS:148-152); olfactory receptors (SEQ ID NOS:156-160); olfactory receptors (SEQ ID NOS:161-180); GPCRs of the human epididymis 6 (HE6), secretin, and latrotoxin receptor families (SEQ ID NOS:181-189); GPCRs of the hepta-helical receptor families (SEQ ID NOS:190-194); mammalian alpha macroglobulin proteins (uses for which are described in, for example, U.S. Pat. No. 5,902,787), complement proteins and cytochrome oxidases (SEQ ID NO:195-199); olfactory receptors, SLIT proteins, LIG-1 protein, and insulin-like growth factor binding proteins (SEQ ID NOS:200-204); taste receptors; see, e.g., GenBank Accession Numbers AAS97395, 20142349 and DAA00013 (SEQ ID NOS:205-221); and the mammalian TRK-fused gene, latrophilin (latrotoxin receptor), and peptide hormone receptors (SEQ ID NOS:232-250).

While certain of the disclosed sequences display sequence and structural homology to mammalian olfactory receptors, the inventors have discovered that these sequences can be expressed in a variety of human cells beyond olfactory receptors, which indicate that the described receptors can play important biological roles in the body beyond that of olfactory functions.

The invention encompasses the use of the described membrane protein nucleotides, membrane proteins and peptides, antibodies, preferably humanized monoclonal antibodies, or binding fragments, domains, or fusion proteins thereof, to the disclosed membrane proteins (which can, for example, act as agonists or antagonists of the disclosed membrane proteins), and antagonists that inhibit activity or expression of the disclosed membrane proteins, or agonists that increase or activate activity or expression of the disclosed membrane proteins, in diagnosis and/or treatment of disease.

In particular, the invention described in the subsections below encompasses polypeptides or peptides corresponding to functional domains of the disclosed membrane proteins (e.g., ECD, TM or CD), mutated, truncated or deleted versions of the disclosed membrane proteins (e.g., missing one or more functional domains or portions thereof, such as, ΔECD, ΔTM and/or ΔCD), fusion proteins (e.g., a membrane protein or a functional domain of a membrane protein, such as the ECD, fused to an unrelated protein or peptide such as an immunoglobulin constant region, i.e., IgFc), nucleotide sequences encoding such products, and host cell expression systems that can produce such products.

The invention also encompasses antibodies and anti-idiotypic antibodies (including Fab fragments), antagonists and agonists of the disclosed membrane proteins, as well as compounds or nucleotide constructs that inhibit expression (transcription factor inhibitors, antisense and ribozyme molecules, or gene or regulatory sequence replacement constructs), or promote expression (e.g., expression constructs in which membrane protein coding sequences are operatively associated with expression control elements such as promoters, promoter/enhancers, etc.), of a gene encoding one or more of the disclosed membrane proteins. The invention also relates to host cells and animals genetically engineered to express the disclosed human membrane proteins (or mutants thereof), or to inhibit or “knock-out” expression of the animal's endogenous membrane protein gene(s).

The disclosed proteins or peptides, fusion proteins, nucleotide sequences, antibodies, antagonists, and agonists, can be useful for the detection of mutant versions of the disclosed membrane proteins, or inappropriately expressed membrane protein sequences, for the diagnosis of disease. The disclosed proteins, peptides, fusion proteins, nucleotide sequences, host cell expression systems, antibodies, antagonists, agonists, and genetically engineered cells and animals, can be used for screening for drugs (or high throughput screening of combinatorial libraries) effective in the treatment of the symptomatic or phenotypic manifestations of perturbing the normal function of the membrane protein in the body. In the case of GPCRs, the use of engineered host cells and/or animals may offer an advantage in that such systems allow not only for the identification of compounds that bind to an ECD of a GPCR, but can also identify compounds that affect the signal transduced by an activated GPCR.

Finally, the disclosed protein products (especially soluble derivatives such as peptides corresponding to an ECD, or truncated polypeptides lacking one or more TM domains), fusion protein products (especially Ig fusion proteins, i.e., fusions of a membrane protein, or a domain of a membrane protein (e.g., ECD or ΔTM) to an IgFc), antibodies and anti-idiotypic antibodies (including Fab fragments), antagonists or agonists (including compounds that modulate signal transduction, which may act on downstream targets in a GPCR- or membrane protein-mediated signal transduction pathway), can be used for therapy of such diseases. For example, the administration of an effective amount of a soluble ECD, ΔTM, or an ECD-IgFc fusion protein, or an anti-idiotypic antibody (or its Fab) that mimics the membrane protein ECD, would “mop up” or “neutralize” the endogenous membrane protein ligand, and prevent or reduce binding and receptor activation.

Nucleotide constructs encoding such membrane protein products can be used to genetically engineer host cells to express such products in vivo; these genetically engineered cells function as “bioreactors” in the body, delivering a continuous supply of a membrane protein, or peptide thereof, soluble ECD or ΔTM, or a membrane protein fusion protein, that will “mop up” or neutralize a membrane protein ligand. Nucleotide constructs encoding functional membrane proteins, mutant membrane proteins, as well as antisense and ribozyme molecules, can be used in “gene therapy” approaches for the modulation of membrane protein expression. Thus, the invention also encompasses pharmaceutical formulations and methods for treating biological disorders.

Various aspects of the invention are described in greater detail in the subsections below.

### 7.1 Membrane Protein Nucleic Acid Sequences

The cDNA sequences and deduced amino acid sequences of the described human membrane proteins (SEQ ID NOS:1-257) are presented in the Sequence Listing.

A number of polymorphisms were identified during the sequencing of the disclosed nucleic acid sequences, including: a G/A polymorphism at nucleotide (nt) position 817 of SEQ ID NO:1 (denoted by an “r” in the Sequence Listing), which can result in a valine or isoleucine residue at corresponding amino acid (aa) position 273 of SEQ ID NO:2; a G/A polymorphism at nt position 923 of SEQ ID NO:1 (denoted by an “r” in the Sequence Listing), which can result in an arginine or histidine residue at corresponding aa position 308 of SEQ ID NO:2; a C/T polymorphism at nt position 320 of SEQ ID NO:132 and nt position 86 of SEQ ID NO:134 (denoted by a “y” in the Sequence Listing), which can result in a serine (preferred) or phenylalanine residue at corresponding aa position 107 of SEQ ID NO:133 and aa position 29 of SEQ ID NO:135; an A/G polymorphism at nt position 1114 of SEQ ID NO:132, nt position 880 of SEQ ID NO:134, and nt position 520 of SEQ ID NO:136 (denoted by an “r” in the Sequence Listing”), which can result in an alanine (preferred) or threonine residue at corresponding aa position 372 of SEQ ID NO:133, aa position 294 of SEQ ID NO:135, and aa position 174 of SEQ ID NO:137; a silent C/T polymorphism at nt position 2283 of SEQ ID NO:132, nt position 2049 of SEQ ID NO:134, and nt position 462 of SEQ ID NO:138, both of which lead to a cysteine residue at corresponding aa position 761 of SEQ ID NO:133, aa position 683 of SEQ ID NO:135, and aa position 154 of SEQ ID NO:139; a silent G/C/T polymorphism at nt position 474 of SEQ ID NO:145 (denoted by a “b” in the Sequence Listing), each of which results in a glycine residue at corresponding aa position 158 of SEQ ID NO:146; a G/A polymorphism at nt position 1457 of SEQ ID NOS:148 and 150 (denoted by an “r” in the Sequence Listing), which can result in an arginine or glutamine residue at corresponding aa position 486 of SEQ ID NOS:149 and 151; a G/A polymorphism at nt position 1678 of SEQ ID NOS:148 and 150 (denoted by an “r” in the Sequence Listing), which can result in a lysine or glutamate residue at corresponding aa position 560 of SEQ ID NOS:149 and 151; a G/T polymorphism at nt position 3535 of SEQ ID NO:148 (denoted by a “k” in the Sequence Listing), which can result in a valine or leucine residue at corresponding aa position 1178 of SEQ ID NO:149; a T/G polymorphism at nt position 233 of SEQ ID NO:153, which can result in a valine or glycine residue at corresponding aa position 78 of SEQ ID NO:154; a C/T polymorphism at nt position 316 of SEQ ID NO:153, which can result in an arginine or cysteine residue at corresponding aa position 106 of SEQ ID NO:154; a silent T/C polymorphism at nt position 1401 of SEQ ID NO:156 (denoted by a “y” in the Sequence Listing), both of which result in a histidine residue at corresponding aa position 467 of SEQ ID NO:157; a silent G/A polymorphism at nt position 2430 of SEQ ID NO:156 (denoted by an “r” in the Sequence Listing), both of which result in a proline residue at corresponding aa position 810 of SEQ ID NO:157; a G/A polymorphism at nt position 146 of SEQ ID NO:168, which can result in a serine or asparagine residue at corresponding aa position 49 of SEQ ID NO:169; a silent C/T polymorphism at nt position 511 of SEQ ID NO:171, both of which result in a leucine residue at corresponding aa position 171 of SEQ ID NO:172; a C/A polymorphism at nt position 766 of SEQ ID NO:171, which can result in a leucine or methionine residue at corresponding aa position 256 of SEQ ID NO:172; an A/G polymorphism at nt position 420 of SEQ ID NO:174 and nt position 276 of SEQ ID NO:176, which can result in a leucine or methionine residue at corresponding aa position 140 of SEQ ID NO:175 and aa position 92 of SEQ ID NO:177; a silent T/C polymorphism at nt position 432 of SEQ ID NO:179 (denoted by a “y” in the Sequence Listing), both of which result in an alanine residue at corresponding aa position 144 of SEQ ID NO:180; a T/C polymorphism at nt position 3601 of SEQ ID NO:181 (denoted by a “y” in the Sequence Listing), which can result in a phenylalanine or leucine residue at corresponding aa position 1201 of SEQ ID NO:182; a T/C polymorphism at nt position 2173 of SEQ ID NO:183 (denoted by a “y” in the Sequence Listing), which can result in a phenylalanine or leucine residue at corresponding aa position 725 of SEQ ID NO:184; a G/A polymorphism at nt position 239 of SEQ ID NO:190 and nt position 53 of SEQ ID NO:192, which can result in a glutamine or arginine residue at corresponding aa position 80 of SEQ ID NO:191 and aa position 18 of SEQ ID NO:193; a silent G/T polymorphism at nt position 723 of SEQ ID NO:190 and nt position 537 of SEQ ID NO:192, both of which result in a serine residue at corresponding aa position 241 of SEQ ID NO:191 and aa position 179 of SEQ ID NO:193; an A/T polymorphism at nucleotide position 766 of SEQ ID NO:190 and nt position 580 of SEQ ID NO:192, which can result in a leucine or methionine residue at corresponding aa position 256 of SEQ ID NO:191 and aa position 194 of SEQ ID NO:193; a silent T/C polymorphism at nucleotide position 1074 of SEQ ID NO:190 and nt position 888 of SEQ ID NO:192, both of which result in a serine residue at corresponding aa position 358 of SEQ ID NO:191 and aa position 296 of SEQ ID NO:193; a G/A polymorphism at nucleotide position 1075 of SEQ ID NO:190 and nt position 889 of SEQ ID NO:192, which can result in a glutamate or lysine residue at corresponding aa position 359 of SEQ ID NO:191 and aa position 297 of SEQ ID NO:193; a G/A polymorphism at nucleotide position 1195 of SEQ ID NO:190 and nt position 1009 of SEQ ID NO:192, which can result in an isoleucine or valine residue at corresponding aa position 399 of SEQ ID NO:191 and aa position 337 of SEQ ID NO:193; an A/G polymorphism at nt position 2108 of SEQ ID NOS:195 and 197, which can result in a tyrosine or cysteine residue at corresponding aa position 703 of SEQ ID NOS:196 and 198; an A/G polymorphism at nt position 2390 of SEQ ID NOS:195 and 197, which can result in a serine or asparagine residue at aa position 797 of SEQ ID NOS:196 and 198; a T/C polymorphism at nt position 3722 of SEQ ID NO:195, which can result in a methionine or threonine residue at corresponding aa position 1241 of SEQ ID NO:196; a G/A polymorphism at nucleotide position 2533 of SEQ ID NOS:195 and 197, which can result in a valine or isoleucine residue at aa position 845 of SEQ ID NOS:196 and 198; a silent T/C polymorphism at nucleotide position 4287 of SEQ ID NO:195, both of which result in a valine residue at corresponding aa position 1429 of SEQ ID NO:196; a C/G polymorphism at nt position 216 of SEQ ID NOS:205 and 207, and nt position 102 of SEQ ID NOS:217 and 219, which can result in an aspartate or glutamate residue at corresponding aa position 72 of SEQ ID NOS:206 and 208, and aa position 34 of SEQ ID NOS:218 and 220; a silent G/A polymorphism at nt position 1320 of SEQ ID NOS:205 and 207, nt position 981 of SEQ ID NOS:209 and 213, nt position 975 of SEQ ID NOS:211 and 215, and nt position 1206 of SEQ ID NOS:217 and 219, both of which encode a glutamine residue at corresponding aa position 440 of SEQ ID NOS:206 and 208, aa position 327 of SEQ ID NOS:210 and 214, aa position 325 of SEQ ID NOS:212 and 216, and aa position 402 of SEQ ID NOS:218 and 220; a C/G polymorphism at nt position 1324 of SEQ ID NOS:205 and 207, nt position 985 of SEQ ID NOS:209 and 213, nt position 979 of SEQ ID NOS:211 and 215, and nt position 1210 of SEQ ID NOS:217 and 219, which can result in a leucine or valine residue at corresponding aa position 442 of SEQ ID NOS:206 and 208, aa position 329 of SEQ ID NOS:210 and 214, aa position 327 of SEQ ID NOS:212 and 216, and aa position 404 of SEQ ID NOS:218 and 220; a silent C/T polymorphism at nt position 1347 of SEQ ID NOS:205 and 207, nt position 1008 of SEQ ID NOS:209 and 213, nt position 1002 of SEQ ID NOS:211 and 215, and nt position 1233 of SEQ ID NOS:217 and 219, both of which encode an asparagine residue at corresponding aa position 449 of SEQ ID NOS:206 and 208, aa position 336 of SEQ ID NOS:210 and 214, aa position 334 of SEQ ID NOS:212 and 216, and aa position 411 of SEQ ID NOS:218 and 220; a G/C polymorphism at nt position 1485 of SEQ ID NOS:206 and 208, nt position 1146 of SEQ ID NOS:209 and 213, nt position 1140 of SEQ ID NOS:211 and 215, and nt position 1371 of SEQ ID NOS:217 and 219, which can result in a lysine or asparagine residue at corresponding aa position 495 of SEQ ID NOS:206 and 208, aa position 382 of SEQ ID NOS:210 and 214, aa position 380 of SEQ ID NOS:212 and 216, and aa position 457 of SEQ ID NOS:218 and 220; a G/A polymorphism at nt position 1522 of SEQ ID NOS:205 and 207, nt position 1183 of SEQ ID NOS:209 and 213, nt position 1177 of SEQ ID NOS:211 and 215, and nt position 1408 of SEQ ID NOS:217 and 219, which can result in an aspartate or asparagine residue at corresponding aa position 508 of SEQ ID NO:206 and 208, aa position 395 of SEQ ID NOS:210 and 214, aa position 393 of SEQ ID NOS:212 and 216, and aa position 470 of SEQ ID NOS:218 and 220; a silent C/G polymorphism at nt position 1542 of SEQ ID NOS:205 and 207, nt position 1203 of SEQ ID NOS:209 and 213, nt position 1197 of SEQ ID NOS:211 and 215, and nt position 1428 of SEQ ID NOS:217 and 219, both of which encode a glycine residue at corresponding aa position 514 of SEQ ID NOS:206 and 208, aa position 401 of SEQ ID NOS:210 and 214, aa position 399 of SEQ ID NOS:212 and 216, and aa position 476 of SEQ ID NOS:218 and 220; a G/A polymorphism at nt position 2306 of SEQ ID NO:205, nt position 2330 of SEQ ID NO:207, nt position 1967 of SEQ ID NO:209, nt position 1961 of SEQ ID NO:211, nt position 1991 of SEQ ID NO:213, nt position 1985 of SEQ ID NO:215, nt position 2192 of SEQ ID NO:217, and nt position 2216 of SEQ ID NO:219, which can result in a glycine or aspartate residue at corresponding aa position 769 of SEQ ID NO:206, aa position 777 of SEQ ID NO:208, aa position 656 of SEQ ID NO:210, aa position 654 of SEQ ID NO:212, aa position 664 of SEQ ID NO:214, aa position 662 of SEQ ID NO:216, aa position 731 of SEQ ID NO:218, and aa position 739 of SEQ ID NO:220; a GCG/CCC polymorphism at nt positions 2311-2313 of SEQ ID NO:205, nt positions 2335-2337 of SEQ ID NO:207, nt positions 1972-1974 of SEQ ID NO:209, nt positions 1966-1968 of SEQ ID NO:211, nt positions 1996-1998 of SEQ ID NO:213, nt positions 1990-1992 of SEQ ID NO:215, nt positions 2197-2199 of SEQ ID NO:217, and nt positions 2221-2223 of SEQ ID NO:219, which can result in an alanine or proline residue at corresponding aa position 771 of SEQ ID NO:206, aa position 779 of SEQ ID NO:208, aa position 658 of SEQ ID NO:210, aa position 656 of SEQ ID NO:212, aa position 666 of SEQ ID NO:214, aa position 664 of SEQ ID NO:216, aa position 733 of SEQ ID NO:218, and aa position 741 of SEQ ID NO:220; a C/T polymorphism at nt position 2383 of SEQ ID NO:205, nt position 2407 of SEQ ID NO:207, nt position 2044 of SEQ ID NO:209, nt position 2038 of SEQ ID NO:211, nt position 2068 of SEQ ID NO:213, nt position 2062 of SEQ ID NO:215, nt position 2269 of SEQ ID NO:217, and nt position 2293 of SEQ ID NO:219, which can result in an arginine or cysteine residue at corresponding aa position 795 of SEQ ID NO:206, aa position 803 of SEQ ID NO:208, aa position 682 of SEQ ID NO:210, aa position 680 of SEQ ID NO:212, aa position 690 of SEQ ID NO:214, aa position 688 of SEQ ID NO:216, aa position 757 of SEQ ID NO:218, and aa position 765 of SEQ ID NO:220; a silent C/T polymorphism at nt position 846 of SEQ ID NOS:226 and 228, both of which result in an isoleucine residue at corresponding aa position 282 of SEQ ID NOS:227 and 229; an A/G polymorphism in the 5′ UTR of SEQ ID NOS:230 and 231; a C/T polymorphism at nt position 1075 of SEQ ID NOS:232 and 236, and nt position 211 of SEQ ID NOS:238 and 242, which can result in a glutamine residue (preferred) or a stop codon at corresponding aa position 359 of SEQ ID NOS:233 and 237, and aa position 71 of SEQ ID NOS:239 and 243; and a silent A/G polymorphism at nt position 2091 of SEQ ID NO:251 and nt position 1587 of SEQ ID NO:253 (denoted by an “r” in the Sequence Listing), both of which result in a glutamine residue at corresponding aa position 697 of SEQ ID NO:252, and aa position 529 of SEQ ID NO:254.

The genes encoding the described membrane proteins are apparently located on: a single coding exon (which may include one or more introns that can be spliced-out in certain cells or tissues) on human chromosome 11, see GenBank Accession Number AC027026 (SEQ ID NOS:123-131); human chromosome 6, see GenBank Accession Number AL355518 (SEQ ID NOS:141-144); human chromosome 6 (SEQ ID NOS:156-160); the human X chromosome, see GenBank Accession Number AL161778 (SEQ ID NOS:181-189); human chromosome 6, see GenBank Accession Number AL356421 (SEQ ID NOS:190-194); human chromosome 6, see GenBank Accession Number AC026605 (SEQ ID NOS:195-199); human chromosome 8 and/or 18 (SEQ ID NOS:200-204); and human chromosome 10, see GenBank Accession Number AL139281 (SEQ ID NOS:205-221).

The present invention includes the human DNA sequences presented in the Sequence Listing (and vectors comprising the same), and additionally contemplates any nucleotide sequence encoding a contiguous and functional membrane protein ORF that hybridizes to a complement of a DNA sequences presented in the Sequence Listing under highly stringent conditions, e.g., hybridization to filter-bound DNA in 0.5 M NaHPO₄, 7% sodium dodecyl sulfate (SDS), 1 mM EDTA at 65° C., and washing in 0.1×SSC/0.1% SDS at 68° C. (“Current Protocols in Molecular Biology” (Ausubel et al., eds., Green Publishing Associates, Inc., and John Wiley & Sons, Inc., New York, 1989)) and encodes a functionally equivalent gene product. Additionally contemplated are any nucleotide sequences that hybridize to the complement of DNA sequences that encode and express an amino acid sequence presented in the Sequence Listing under moderately stringent conditions, e.g., washing in 0.2×SSC/0.1% SDS at 42° C. (“Current Protocols in Molecular Biology”, supra), yet that still encode a functionally equivalent gene product. Functional equivalents of the disclosed membrane proteins include naturally occurring membrane proteins present in other species, and mutant membrane proteins, whether naturally occurring or engineered (by site directed mutagenesis, gene shuffling, directed evolution as described in, for example, U.S. Pat. Nos. 5,837,458 and 5,723,323). The invention also includes degenerate nucleic acid variants of the disclosed polynucleotide sequences.

Additionally contemplated are polynucleotides encoding membrane protein ORFs, or their functional equivalents, encoded by polynucleotide sequences that are about 99, 95, 90, or about 85 percent similar or identical to corresponding regions of the polynucleotide sequences described in the Sequence Listing (as measured by BLAST sequence comparison analysis using, for example, the GCG sequence analysis package (SEQUENCHER 3.0, Gene Codes Corp., Ann Arbor, Mich.) using default parameters).

The invention also includes nucleic acid molecules, preferably DNA molecules, that hybridize to, and are therefore the complements of, the described nucleotide sequences. Such hybridization conditions may be highly stringent or less highly stringent, as described herein. In instances wherein the nucleic acid molecules are deoxyoligonucleotides, such molecules are about 16 to about 100 bases long, about 20 to about 80 bases long, or about 34 to about 45 bases long, or any variation or combination of sizes represented therein, incorporating a contiguous region of nucleotide sequence first disclosed in the Sequence Listing, and can be used in conjunction with the polymerase chain reaction (PCR) to screen libraries, isolate clones, and prepare cloning and sequencing templates, etc.

Alternatively, such oligonucleotides can be used as hybridization probes for screening libraries, and assessing gene expression patterns (particularly using a microarray or high-throughput “chip” format). Additionally, a series of the described oligonucleotide sequences, or the complements thereof, can be used to represent all or a portion of the described membrane protein sequences. An oligonucleotide or polynucleotide sequence first disclosed in at least a portion of one or more of the sequences of SEQ ID NOS:1-257 can be used as a hybridization probe in conjunction with a solid support matrix/substrate (resins, beads, membranes, plastics, polymers, metal or metallized substrates, crystalline or polycrystalline substrates, etc.). Of particular note are spatially addressable arrays (i.e., gene chips, microtiter plates, etc.) of oligonucleotides and polynucleotides, or corresponding oligopeptides and polypeptides, wherein at least one of the biopolymers present on the spatially addressable array comprises an oligonucleotide or polynucleotide sequence first disclosed in at least one of the sequences of SEQ ID NOS:1-257, or an amino acid sequence encoded thereby. Methods for attaching biopolymers to, or synthesizing biopolymers on, solid support matrices, and conducting binding studies thereon are disclosed in, inter alia, U.S. Pat. Nos. 5,700,637, 5,556,752, 5,744,305, 4,631,211, 5,445,934, 5,252,743, 4,713,326, 5,424,186, and 4,689,405.

Addressable arrays comprising sequences first disclosed in SEQ ID NOS:1-257 can be used to identify and characterize the temporal and tissue specific expression of a gene. These addressable arrays incorporate oligonucleotide sequences of sufficient length to confer the required specificity, yet be within the limitations of the production technology. The length of these probes is within a range of between about 8 to about 2000 nucleotides. Preferably the probes consist of 60 nucleotides and more preferably 25 nucleotides from the sequences first disclosed in SEQ ID NOS:1-257.

For example, a series of the described oligonucleotide sequences, or the complements thereof, can be used in chip format to represent all or a portion of the described membrane protein sequences. The oligonucleotides, typically between about 16 to about 40 (or any whole number within the stated range) nucleotides in length, can partially overlap each other and/or the membrane protein sequence may be represented using oligonucleotides that do not overlap. Accordingly, the described polynucleotide sequences shall typically comprise at least about two or three distinct oligonucleotide sequences of at least about 18 nucleotides in length that are each first disclosed in the Sequence Listing. Such oligonucleotide sequences can begin at any nucleotide present within a sequence in the Sequence Listing and proceed in either a sense (5′-to-3′) orientation vis-a-vis the described sequence or in an antisense (3′-to-5′) orientation.

Microarray-based analysis allows the discovery of broad patterns of genetic activity, providing new understanding of gene functions and generating novel and unexpected insight into transcriptional processes and biological mechanisms. The use of addressable arrays comprising sequences first disclosed in SEQ ID NOS:1-257 provides detailed information about transcriptional changes involved in a specific pathway, potentially leading to the identification of novel components or gene functions that manifest themselves as novel phenotypes.

Probes consisting of sequences first disclosed in SEQ ID NOS:1-257 can also be used in the identification, selection and validation of novel molecular targets for drug discovery. The use of these unique sequences permits the direct confirmation of drug targets and recognition of drug dependent changes in gene expression that are modulated through pathways distinct from the intended target of the drug. These unique sequences therefore also have utility in defining and monitoring both drug action and toxicity.

As an example of utility, the sequences first disclosed in SEQ ID NOS:1-257 can be utilized in microarrays or other assay formats, to screen collections of genetic material from patients who have a particular medical condition. These investigations can also be carried out using the sequences first disclosed in SEQ ID NOS:1-257 in silico and by comparing previously collected genetic databases and the disclosed sequences using computer software known to those in the art. Thus the sequences first disclosed in SEQ ID NOS:1-257 can be used to identify mutations associated with a particular disease, and also in diagnostic or prognostic assays.

Although the presently described sequences have been specifically described using nucleotide sequence, it should be appreciated that each of the sequences can uniquely be described using any of a wide variety of additional structural attributes, or combinations thereof. For example, a given sequence can be described by the net composition of the nucleotides present within a given region of the sequence in conjunction with the presence of one or more specific oligonucleotide sequence(s) first disclosed in SEQ ID NOS:1-257. Alternatively, a restriction map specifying the relative positions of restriction endonuclease digestion sites, or various palindromic or other specific oligonucleotide sequences can be used to structurally describe a given sequence. Such restriction maps, which are typically generated by widely available computer programs (e.g., SEQUENCHER 3.0, Gene Codes Corp., etc.), can optionally be used in conjunction with one or more discrete nucleotide sequence(s) present in the sequence that can be described by the relative position of the sequence relative to one or more additional sequence(s) or one or more restriction sites present in the disclosed sequence.

For oligonucleotides probes, highly stringent conditions may refer, e.g., to washing in 6×SSC/0.05% sodium pyrophosphate at 37° C. (for 14-base oligos), 48° C. (for 17-base oligos), 55° C. (for 20-base oligos), and 60° C. (for 23-base oligos). The described oligonucleotides may encode or act as antisense molecules, useful, for example, in gene regulation (and/or as antisense primers in amplification reactions of membrane protein nucleic acid sequences). With respect to gene regulation, such techniques can be used to regulate biological functions. Further, such sequences may be used as part of ribozyme and/or triple helix sequences, also useful for gene regulation.

Additionally, the antisense oligonucleotides may comprise at least one modified base moiety that is selected from the group including, but not limited to, 5-fluorouracil, 5-bromouracil, 5-chlorouracil, 5-iodouracil, hypoxanthine, xanthine, inosine, 4-acetylcytosine, 5-(carboxyhydroxylmethyl) uracil, dihydrouracil, 5-carboxymethylaminomethyl-2-thiouridine, 5-carboxymethylaminomethyluracil, beta-D-galactosylqueosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 2-thiouracil, 4-thiouracil, 5-methyluracil, uracil-5-oxyacetic acid methylester, 5-methyl-2-thiouracil, 3-(3-amino-3-N-2-carboxypropyl) uracil, (acp3)w, and 2,6-diaminopurine.

The antisense oligonucleotide may also comprise at least one modified sugar moiety selected from the group including, but not limited to, arabinose, 2-fluoroarabinose, xylulose, and hexose. In another embodiment, the antisense oligonucleotide comprises at least one modified phosphate backbone selected from the group consisting of a phosphorothioate, a phosphorodithioate, a phosphoramidothioate, a phosphoramidate, a phosphordiamidate, a methylphosphonate, an alkyl phosphotriester, and a formacetal or analog thereof. In yet another embodiment, the antisense oligonucleotide is an α-anomeric oligonucleotide. An α-anomeric oligonucleotide forms specific double-stranded hybrids with complementary RNA in which, contrary to the usual β-units, the strands run parallel to each other (Gautier et al., Nucl. Acids Res. 15:6625-6641, 1987). The oligonucleotide is a 2′-0-methylribonucleotide (Inoue et al., Nucl. Acids Res. 15:6131-6148, 1987), or a chimeric RNA-DNA analogue (Inoue et al., FEBS Lett. 215:327-330, 1987). Alternatively, dsRNA can be used to disrupt the expression and function of a targeted membrane protein sequence.

Oligonucleotides of the invention may be synthesized by standard methods known in the art, e.g., by use of an automated DNA synthesizer (such as are commercially available from Biosearch Technologies, Inc. (Novato, Calif.), Applied Biosystems (Foster City, Calif.), etc.). As examples, phosphorothioate oligonucleotides may be synthesized (Stein et al., Nucl. Acids Res. 16:3209-3221, 1988), and methylphosphonate oligonucleotides can be prepared by use of controlled pore glass polymer supports (Sarin et al., Proc. Natl. Acad. Sci. USA 85:7448-7451, 1988).

Low stringency conditions are well-known to those of skill in the art, and will vary predictably depending on the specific organisms from which the library and the labeled sequences are derived. For guidance regarding such conditions, see, e.g., “Molecular Cloning, A Laboratory Manual” (Sambrook et al., eds., Cold Spring Harbor Press, Cold Spring Harbor, N.Y., 1989), “Current Protocols in Molecular Biology”, supra, and periodic updates thereof.

Alternatively, suitably labeled nucleotide probes may be used to screen a human genomic library using appropriately stringent conditions, or by PCR. The identification and characterization of human genomic clones is helpful for identifying polymorphisms (including, but not limited to, nucleotide repeats, microsatellite alleles, single nucleotide polymorphisms, or coding single nucleotide polymorphisms), determining the genomic structure of a given locus/allele, and designing diagnostic tests. For example, sequences derived from regions adjacent to the intron/exon boundaries of the human gene can be used to design primers for use in amplification assays to detect mutations within the exons, introns, splice sites (e.g., splice acceptor and/or donor sites), etc., that can be used in diagnostics and pharmacogenomics.

For example, the present sequences can be used in RFLP analysis to identify specific individuals. In this technique, an individual's genomic DNA is digested with one or more restriction enzymes, and probed on a Southern blot to yield unique bands for identification (as generally described in U.S. Pat. No. 5,272,057). In addition, the sequences of the present invention can be used to provide polynucleotide reagents, e.g., PCR primers, targeted to specific loci in the human genome, which can enhance the reliability of DNA-based forensic identifications by, for example, providing another “identification marker” (i.e., another DNA sequence that is unique to a particular individual). Actual base sequence information can be used for identification as an accurate alternative to patterns formed by restriction enzyme generated fragments.

Further, homologs of the disclosed membrane protein sequences may be isolated from nucleic acids of the organism of interest by performing PCR using two degenerate oligonucleotide primer pools designed on the basis of amino acid sequences within the membrane proteins or gene products disclosed herein. The template for the reaction may be total RNA, mRNA, and/or cDNA obtained by reverse transcription of mRNA prepared from, for example, human or non-human cell lines or tissue(s) known to express, or suspected of expressing, a membrane protein gene.

The PCR product may be subcloned and sequenced to ensure that the amplified sequences represent the sequence of the desired membrane protein gene. The PCR fragment may then be used to isolate a full length CDNA clone by a variety of methods. For example, the amplified fragment may be labeled and used to screen a cDNA library, such as a bacteriophage cDNA library. Alternatively, the labeled fragment may be used to isolate genomic clones via the screening of a genomic library.

PCR technology may also be utilized to isolate full length cDNA sequences. For example, RNA may be isolated, following standard procedures, from an appropriate cellular or tissue source (i.e., one known to express, or suspected of expressing, a membrane protein gene). A reverse transcription (RT) reaction may be performed on the RNA using an oligonucleotide primer specific for the most 5′ end of the amplified fragment for the priming of first strand synthesis. The resulting RNA/DNA hybrid may then be “tailed” using a standard terminal transferase reaction, the hybrid digested with RNase H, and second strand synthesis primed with a complementary primer. Thus, cDNA sequences upstream of the amplified fragment may easily be isolated. For a review of cloning strategies that may be used, see, e.g., “Molecular Cloning, A Laboratory Manual”, supra.

A cDNA of a mutant membrane protein gene can be isolated, for example, by using PCR. In this case, the first cDNA strand may be synthesized by hybridizing an oligo-dT oligonucleotide to mRNA isolated from tissue known to express, or suspected of expressing, a mutant membrane protein allele, in an individual putatively carrying a mutant membrane protein allele, and by extending the new strand with reverse transcriptase. The second strand of the cDNA is then synthesized using an oligonucleotide that hybridizes specifically to the 5′ end of the normal gene. Using these two primers, the product is then amplified via PCR, optionally cloned into a suitable vector, and subjected to DNA sequence analysis through methods well-known to those of skill in the art. By comparing the DNA sequence of the mutant membrane protein allele to that of the normal membrane protein allele, the mutation(s) responsible for the loss or alteration of function of the mutant membrane protein gene product can be ascertained.

Alternatively, a genomic library can be constructed using DNA obtained from an individual suspected of carrying, or known to carry, a mutant membrane protein allele, or a cDNA library can be constructed using RNA from a tissue known to express, or suspected of expressing, a mutant membrane protein allele. A normal membrane protein gene, or any suitable fragment thereof, can then be labeled and used as a probe to identify the corresponding mutant membrane protein allele in such libraries. Clones containing the mutant membrane protein gene sequences can then be purified and subjected to sequence analysis according to methods well-known to those of skill in the art.

Additionally, an expression library can be constructed utilizing cDNA synthesized from, for example, RNA isolated from a tissue known to express, or suspected of expressing, a mutant membrane protein allele in an individual suspected of carrying, or known to carry, such a mutant allele. In this manner, gene products made by the putatively mutant tissue may be expressed and screened using standard antibody screening techniques in conjunction with antibodies raised against the normal membrane protein, as described below in Section 7.3 (for screening techniques, see, e.g., “Antibodies: A Laboratory Manual” (Harlow and Lane, eds., Cold Spring Harbor Press, Cold Spring Harbor, N.Y., 1988)).

Additionally, screening can be accomplished by screening with labeled membrane protein fusion proteins, such as, for example, alkaline phosphatase-membrane protein or membrane protein-alkaline phosphatase fusion proteins. In cases where a mutation results in an expressed gene product with altered function (e.g., as a result of a missense or a frameshift mutation), a polyclonal set of antibodies to the wild-type membrane protein are likely to cross-react with the mutant membrane protein. Library clones detected via their reaction with such labeled antibodies can be purified and subjected to sequence analysis according to methods well-known to those of skill in the art.

The invention also encompasses nucleotide sequences that encode mutant membrane proteins, peptide fragments of the membrane proteins, truncated membrane proteins, and membrane protein fusion proteins. These include, but are not limited to, nucleotide sequences encoding mutant membrane proteins, as described herein, polypeptides or peptides corresponding to one or more ECD, TM and/or CD domain(s) of a membrane protein (or portions of these domains), truncated membrane proteins in which one or two of the domains is deleted, e.g., a soluble membrane protein lacking the TM or both the TM and CD regions, or a truncated, nonfunctional membrane protein lacking all or a portion of the CD region. Nucleotides encoding fusion proteins may include, but are not limited to, full length membrane protein sequences, truncated membrane proteins, or nucleotides encoding peptide fragments of the disclosed membrane proteins, fused to an unrelated protein or peptide, such as, for example, a transmembrane sequence, which anchors a membrane protein ECD to the cell, an IgFc domain, which increases the stability and half life of the resulting fusion protein in the bloodstream, or an enzyme, fluorescent protein, or luminescent protein that can be used as a marker.

The invention also encompasses: (a) DNA vectors that contain any of the foregoing membrane protein coding sequences and/or their complements (i.e., antisense); (b) DNA expression vectors that contain any of the foregoing membrane protein coding sequences operatively associated with at least a first regulatory element that directs the expression of the coding sequences (for example, baculovirus vectors as described in U.S. Pat. No. 5,869,336); (c) genetically engineered host cells that contain any of the foregoing membrane protein coding sequences operatively associated with at least a first regulatory element that directs the expression of the coding sequences in the host cell; and (d) genetically engineered host cells that express an endogenous membrane protein sequence under the control of an exogenously introduced regulatory element (i.e., gene activation). As used herein, regulatory elements include, but are not limited to, inducible and non-inducible promoters, enhancers, operators, and other elements known to those skilled in the art that drive and regulate expression. Such regulatory elements include, but are not limited to, the cytomegalovirus hCMV immediate early gene, regulatable, viral (particularly retroviral LTR promoters), the early or late promoters of SV40 or adenovirus, the lac system, the trp system, the tet system, the TAC system, the TRC system, the major operator and promoter regions of phage lambda, the control regions of fd coat protein, the promoter for 3-phosphoglycerate kinase, the promoters of acid phosphatase, and the promoters of the yeast α-mating factors.

An additional application of the described membrane protein polynucleotide sequences is their use in the molecular mutagenesis/evolution of proteins that are at least partially encoded by the described novel sequences using, for example, polynucleotide shuffling or related methodologies (see, e.g., U.S. Pat. Nos. 5,830,721, 5,723,323, and 5,837,458).

Additionally contemplated uses for the described sequences include the engineering of constitutively “on” variants for use in cell assays and genetically engineered animals, using the methods and applications described in U.S. Provisional Patent Application Ser. Nos. 60/110,906, 60/106,300, 60/094,879, and 60/121,851.

The disclosed gene products can also be expressed in non-human transgenic animals. Animals of any species, including, but not limited to, worms, mice, rats, rabbits, guinea pigs, pigs, micro-pigs, birds, goats, and non-human primates, e.g., baboons, monkeys, and chimpanzees, may be used to generate transgenic animals.

Any technique known in the art may be used to introduce a membrane protein transgene into animals to produce the founder lines of transgenic animals. Such techniques include, but are not limited to, pronuclear microinjection (U.S. Pat. No. 4,873,191); retrovirus mediated gene transfer into germ lines (Van der Putten et al., Proc. Natl. Acad. Sci. USA 82:6148-6152, 1985); gene targeting in embryonic stem cells (Thompson et al., Cell 56:313-321, 1989); electroporation of embryos (Lo, Mol. Cell. Biol. 3:1803-1814, 1983); and sperm-mediated gene transfer (Lavitrano et al., Cell 57:717-723, 1989); etc. For a review of such techniques, see, e.g., Gordon, Intl. Rev. Cytol. 115:171-229, 1989.

The present invention provides for transgenic animals that carry the membrane protein transgene in all their cells, as well as animals that carry the transgene in some, but not all their cells, i.e., mosaic animals or somatic cell transgenic animals. The transgene may be integrated as a single transgene or in concatamers, e.g., head-to-head tandems or head-to-tail tandems. The transgene may also be selectively introduced into and activated in a particular cell-type by following, for example, the teaching of Lasko et al. (Proc. Natl. Acad. Sci. USA 89:6232-6236, 1992). The regulatory sequences required for such a cell-type specific activation will depend upon the particular cell-type of interest, and will be apparent to those of skill in the art.

When it is desired that a membrane protein transgene be integrated into the chromosomal site of the endogenous membrane protein gene, gene targeting is preferred. Briefly, when such a technique is to be utilized, vectors containing some nucleotide sequences homologous to the endogenous membrane protein gene are designed for the purpose of integrating, via homologous recombination with chromosomal sequences, into and disrupting the function of the nucleotide sequence of the endogenous membrane protein gene (i.e., “knock-out” animals).

The transgene can also be selectively introduced into a particular cell-type, thus inactivating the endogenous membrane protein gene in only that cell-type, by following, for example, the teaching of Gu et al. (Science 265:103-106, 1994). The regulatory sequences required for such a cell-type specific inactivation will depend upon the particular cell-type of interest, and will be apparent to those of skill in the art.

Once transgenic animals have been generated, the expression of the recombinant membrane protein gene may be assayed utilizing standard techniques. Initial screening may be accomplished by Southern blot analysis or PCR techniques to analyze animal tissues to assay whether integration of the transgene has taken place. The level of mRNA expression of the transgene in the tissues of the transgenic animals may also be assessed using techniques that include, but are not limited to, Northern blot analysis of tissue samples obtained from the animal, in situ hybridization analysis, and RT-PCR. Samples of membrane protein gene-expressing tissue may also be evaluated immunocytochemically using antibodies specific for the membrane protein transgene product.

### 7.2 Membrane Protein Amino Acid Sequences

The disclosed membrane proteins, polypeptides, peptide fragments, mutated, truncated or deleted forms of the membrane proteins, and/or fusion proteins can be prepared for a variety of uses, including, but not limited to, as protein therapeutics, in the generation of antibodies, as reagents in diagnostic assays, in the identification of other cellular gene products related to a membrane protein, as reagents in assays for screening for compounds that can be used as pharmaceutical reagents useful in the therapeutic treatment of mental, biological, or medical disorders (i.e., kidney disorders, digestive disorders, infertility, improper blood pressure, body weight disorders, etc.) and disease. Given the similarity information and expression data, the described membrane proteins can be targeted (by drugs, oligos, antibodies, etc.) in order to treat disease, or to therapeutically augment the efficacy of therapeutic agents.

The Sequence Listing discloses the amino acid sequences encoded by the described membrane protein nucleotide sequences. The membrane protein sequences have initiator methionines in DNA sequence contexts consistent with translation initiation sites, in most cases followed by hydrophobic signal sequences typical of membrane-associated proteins or secreted proteins. SEQ ID NO:146 displays an excellent signal sequence, which indicates that this protein may be secreted, membrane-associated, or cytoplasmic, while SEQ ID NOS:149 and 151 display several transmembrane domains, but apparently lack a strong N-terminal signal sequence. SEQ ID NO:154 has an atypical hydrophobic leader sequence. The sequence data presented herein also indicates that alternatively spliced forms of many of the disclosed the membrane proteins exist (which may or may not be tissue specific).

The amino acid sequences of the invention include the amino acid sequences presented in the Sequence Listing, as well as analogues and derivatives thereof. Further, corresponding membrane protein homologues from other species are encompassed by the invention. In fact, any membrane protein encoded by the membrane protein nucleotide sequences described herein are within the scope of the invention, as are any novel polynucleotide sequences encoding all, or any novel portion, of an amino acid sequence presented in the Sequence Listing. The degenerate nature of the genetic code is well-known, and, accordingly, each amino acid presented in the Sequence Listing is generically representative of the well-known nucleic acid “triplet” codon, or in many cases codons, that can encode the amino acid. As such, as contemplated herein, the amino acid sequences presented in the Sequence Listing, when taken together with the genetic code (see, e.g., “Molecular Cell Biology”, Table 4-1 at page 109 (Darnell et al., eds., Scientific American Books, New York, N.Y., 1986)), are generically representative of all the various permutations and combinations of nucleic acid sequences that can encode such amino acid sequences.

The invention also encompasses proteins that are functionally equivalent to the membrane proteins encoded by the described nucleotide sequences as judged by any of a number of criteria, including, but not limited to, the ability to bind a ligand of a membrane protein, the ability to effect an identical or complementary signal transduction pathway, a change in cellular metabolism (e.g., ion flux, tyrosine phosphorylation, etc.), or a change in phenotype when the membrane protein equivalent is present in an appropriate cell-type (such as the amelioration, prevention, or delay of a biochemical, biophysical, or overt phenotype). Such functionally equivalent membrane proteins include, but are not limited to, additions or substitutions of amino acid residues within the amino acid sequences encoded by the membrane protein nucleotide sequences described herein, but that result in a silent change, thus producing a functionally equivalent gene product. Amino acid substitutions may be made on the basis of similarity in polarity, charge, solubility, hydrophobicity, hydrophilicity, and/or the amphipathic nature of the residues involved. For example, nonpolar (hydrophobic) amino acids include alanine, leucine, isoleucine, valine, proline, phenylalanine, tryptophan, and methionine; polar neutral amino acids include glycine, serine, threonine, cysteine, tyrosine, asparagine, and glutamine; positively charged (basic) amino acids include arginine, lysine, and histidine; and negatively charged (acidic) amino acids include aspartic acid and glutamic acid.

While random mutations can be made to a membrane protein DNA (using random mutagenesis techniques well-known to those skilled in the art), and the resulting mutant membrane proteins tested for activity, site-directed mutations of the membrane protein coding sequence can be engineered (using site-directed mutagenesis techniques well-known to those skilled in the art) to generate mutant membrane proteins with increased function (e.g., higher binding affinity for the target ligand and/or greater signaling capacity) or decreased function (e.g., weaker binding affinity for the target ligand and/or decreased signal transduction capacity). One starting point for such analysis is by aligning the disclosed human sequences with corresponding gene/protein sequences from other mammals, for example, in order to identify amino acid sequence motifs that are conserved between different species. Non-conservative changes can be engineered at variable positions to alter function, signal transduction capability, or both. Alternatively, where alteration of function is desired, deletion or non-conservative alterations of the conserved regions (i.e., identical amino acids) can be engineered, for example, deletion or non-conservative alterations (substitutions or insertions) of the various conserved transmembrane domains.

Other mutations in the membrane protein coding sequence can be made to generate membrane proteins that are better suited for expression, scale up, etc., in the host cells chosen. For example, cysteine residues can be deleted or substituted with another amino acid in order to eliminate disulfide bridges, and N-linked glycosylation sites can be altered or eliminated to achieve, for example, expression of a homogeneous product that is more easily recovered and purified from yeast hosts, which are known to hyperglycosylate N-linked sites. To this end, a variety of amino acid substitutions at one or both of the first or third amino acid positions of any one or more of the glycosylation recognition sequences that occur in the ECD (N-X-S or N-X-T), and/or an amino acid deletion at the second position of any one or more such recognition sequences in the ECD, will prevent glycosylation of a membrane protein at the modified tripeptide sequence (see, e.g., Miyajima et al., EMBO J. 5:1193-1197, 1986).

Peptides corresponding to one or more domains of the membrane proteins (e.g., ECD, TM, CD, etc.), truncated or deleted membrane proteins (e.g., membrane proteins in which an ECD, TM, and/or CD is deleted), as well as fusion proteins in which a full length membrane protein, a membrane protein peptide, or truncated membrane protein is fused to an unrelated protein, are also within the scope of the invention, and can be designed on the basis of the presently disclosed nucleotide and amino acid sequences. Such fusion proteins include, but are not limited to: IgFc fusions, which stabilize the membrane protein or peptide and prolong half-life in vivo; fusions to any amino acid sequence that allows the fusion protein to be anchored to the cell membrane, allowing an ECD to be exhibited on the cell surface; or fusions to an enzyme, fluorescent protein, or luminescent protein that provides a marker function.

While the membrane proteins, polypeptides, and peptides can be chemically synthesized (see, e.g., “Proteins: Structures and Molecular Principles” (Creighton, ed., W. H. Freeman & Co., New York, N.Y., 1983)), large polypeptides derived from a membrane protein and full length membrane proteins can be advantageously produced by recombinant DNA technology using techniques well-known in the art for expressing membrane protein gene and/or coding sequences. Such methods can be used to construct expression vectors containing one or more of the presently described membrane protein nucleotide sequences and appropriate transcriptional and translational control signals. These methods include, for example, in vitro recombinant DNA techniques, synthetic techniques, and in vivo genetic recombination (see, e.g., the techniques described in “Molecular Cloning, A Laboratory Manual”, supra, and “Current Protocols in Molecular Biology”, supra). Alternatively, RNA corresponding to all or a portion of a transcript encoded by a membrane protein nucleotide sequence may be chemically synthesized using, for example, synthesizers (see, e.g., the techniques described in “Oligonucleotide Synthesis” (Gait, ed., IRL Press, Oxford, 1984)).

A variety of host-expression vector systems may be utilized to express the nucleotide sequences of the invention. Where the peptide or polypeptide of the membrane protein is a soluble derivative (e.g., peptides corresponding to an ECD, or truncated or deleted membrane proteins in which a TM and/or CD are deleted), the peptide or polypeptide can be recovered from the culture, i.e., from the host cell in cases where the peptide or polypeptide is not secreted, and from the culture media in cases where the peptide or polypeptide is secreted by the host cell. However, such expression systems also encompass engineered host cells that express a membrane protein, or functional equivalent, in situ, i.e., anchored in the cell membrane. Purification or enrichment of membrane proteins from such expression systems can be accomplished using appropriate detergents and lipid micelles and methods well-known to those skilled in the art. However, such engineered host cells themselves may be used in situations where it is important not only to retain the structural and functional characteristics of the membrane protein, but to assess biological activity, e.g., in certain drug screening assays.

The expression systems that may be used for purposes of the invention include, but are not limited to, microorganisms such as bacteria (e.g., E. coli, B. subtilis) transformed with recombinant bacteriophage DNA, plasmid DNA or cosmid DNA expression vectors containing membrane protein nucleotide sequences; yeast (e.g., Saccharomyces, Pichia) transformed with recombinant yeast expression vectors containing membrane protein nucleotide sequences; insect cell systems infected with recombinant virus expression vectors (e.g., baculovirus) containing membrane protein nucleotide sequences; plant cell systems infected with recombinant virus expression vectors (e.g., cauliflower mosaic virus, CaMV; tobacco mosaic virus, TMV) or transformed with recombinant plasmid expression vectors (e.g., Ti plasmid) containing membrane protein nucleotide sequences; or mammalian cell systems (e.g., COS, CHO, BHK, 293, 3T3) harboring recombinant expression constructs containing membrane protein nucleotide sequences and promoters derived from the genome of mammalian cells (e.g., metallothionein promoter) or from mammalian viruses (e.g., the adenovirus late promoter or the vaccinia virus 7.5K promoter).

In bacterial systems, a number of expression vectors may be advantageously selected depending upon the use intended for the membrane protein gene product being expressed. For example, when a large quantity of such a protein is to be produced, such as in the generation of pharmaceutical compositions of a membrane protein or for raising antibodies to a membrane protein, vectors that direct the expression of high levels of fusion protein products that are readily purified may be desirable. Such vectors include, but are not limited to, the E. coli expression vector pUR278 (Ruther and Muller-Hill, EMBO J. 2:1791-1794, 1983), in which a membrane protein coding sequence may be ligated individually into the vector in frame with the lacZ coding region so that a fusion protein is produced; pIN vectors (Inouye and Inouye, Nucl. Acids Res. 13:3101-3109, 1985; Van Heeke and Schuster, J. Biol. Chem. 264:5503-5509, 1989); and the like. pGEX vectors may also be used to express membrane proteins, polypeptides, or peptides, as fusion proteins with glutathione S-transferase (GST) . In general, such fusion proteins are soluble and can easily be purified from lysed cells by adsorption to glutathione-agarose beads followed by elution in the presence of free glutathione. The PGEX vectors are designed to include thrombin or factor Xa protease cleavage sites so that the membrane protein, polypeptide, or peptide, can be released from the GST moiety.

In an exemplary insect system, Autographa californica nuclear polyhedrosis virus (AcNPV) is used as a vector to express membrane protein nucleotide sequences. The virus grows in Spodoptera frugiperda cells. A membrane protein coding sequence may be cloned individually into a non-essential region (for example the polyhedrin gene) of the virus and placed under control of an AcNPV promoter (for example the polyhedrin promoter). Successful insertion of a membrane protein coding sequence will result in inactivation of the polyhedrin gene and production of non-occluded recombinant virus (i.e., virus lacking the proteinaceous coat coded for by the polyhedrin gene). These recombinant viruses are then used to infect Spodoptera frugiperda cells in which the inserted gene is expressed (see, e.g., Smith et al., J. Virol. 46:584-593, 1983, and U.S. Pat. No. 4,215,051).

In mammalian host cells, a number of viral-based expression systems may be utilized. In cases where an adenovirus is used as an expression vector, a membrane protein nucleotide sequence may be ligated to an adenovirus transcription/translation control complex, e.g., the late promoter and tripartite leader sequence. This chimeric sequence may then be inserted in the adenovirus genome by in vitro or in vivo recombination. Insertion in a non-essential region of the viral genome (e.g., region E1 or E3) will result in a recombinant virus that is viable and capable of expressing a membrane protein sequence in infected hosts (see, e.g., Logan and Shenk, Proc. Natl. Acad. Sci. USA 81:3655-3659, 1984). Specific initiation signals may also be required for efficient translation of inserted membrane protein nucleotide sequences. These signals include the ATG initiation codon and adjacent sequences. In cases where an entire membrane protein gene or cDNA, including its own initiation codon and adjacent sequences, is inserted into an appropriate expression vector, no additional translational control signals may be needed. However, in cases where only a portion of a membrane protein coding sequence is inserted, exogenous translational control signals, including, perhaps, the ATG initiation codon, may be provided. Furthermore, the initiation codon should be in phase with the reading frame of the desired membrane protein coding sequence to ensure translation of the entire insert. These exogenous translational control signals and initiation codons can be of a variety of origins, both natural and synthetic. The efficiency of expression may be enhanced by the inclusion of appropriate transcription enhancer elements, transcription terminators, etc. (see, e.g., Bitter et al., Methods in Enzymol. 153:516-544, 1987).

In addition, a host cell strain may be chosen that modulates the expression of the inserted sequences, or modifies and processes the expression product, in the specific fashion desired. Such modifications (e.g., glycosylation) and processing (e.g., cleavage) of protein products may be important for the function of the protein. Different host cells have characteristic and specific mechanisms for the post-translational processing and modification of proteins and gene or expression products. Appropriate cell lines or host systems can be chosen to ensure the correct modification and processing of the foreign protein expressed. To this end, eukaryotic host cells that possess the cellular machinery for proper processing of the primary transcript, glycosylation, and phosphorylation of the gene product may be used. Such mammalian host cells include, but are not limited to, CHO, VERO, BHK, HeLa, COS, MDCK, 293, 3T3, and WI38 cell lines.

For long-term, high-yield production of recombinant proteins, stable expression is preferred. For example, cell lines that stably express the membrane protein sequences described herein may be engineered. Rather than using expression vectors that contain viral origins of replication, host cells can be transformed with DNA controlled by appropriate expression control elements (e.g., promoter, enhancer sequences, transcription terminators, polyadenylation sites, etc.), and a selectable marker. Following the introduction of the foreign DNA, engineered cells may be allowed to grow for 1-2 days in an enriched media, and then are switched to a selective media. The selectable marker in the recombinant plasmid confers resistance to the selection and allows cells to stably integrate the plasmid into their chromosomes and grow to form foci, which in turn can be cloned and expanded into cell lines. This method may advantageously be used to engineer cell lines that express membrane protein sequences. Such engineered cell lines may be particularly useful in screening and evaluation of compounds that affect the endogenous activity of a membrane protein.

A number of selection systems may be used, including, but not limited to, the herpes simplex virus thymidine kinase (Wigler et al., Cell 11:223-232, 1977), hypoxanthine-guanine phosphoribosyltransferase (Szybalska and Szybalski, Proc. Natl. Acad. Sci. USA 48:2026-2034, 1962), and adenine phosphoribosyltransferase (Lowy et al., Cell 22:817-823, 1980) genes, which can be employed in tk⁻, hgprt⁻ or aprt⁻ cells, respectively. Also, antimetabolite resistance can be used as the basis of selection for the following genes: dihydrofolate reductase (dhfr), which confers resistance to methotrexate (Wigler et al., Proc. Natl. Acad. Sci. USA 77:3567-3570, 1980, and O'Hare et al., Proc. Natl. Acad. Sci. USA 78:1527-1531, 1981); guanine phosphoribosyl transferase (gpt), which confers resistance to mycophenolic acid (Mulligan and Berg, Proc. Natl. Acad. Sci. USA 78:2072-2076, 1981); neomycin phosphotransferase (neo), which confers resistance to G-418 (Colbere-Garapin et al., J. Mol. Biol. 150:1-14, 1981); and hygromycin B phosphotransferase (hpt), which confers resistance to hygromycin (Santerre et al., Gene 30:147-156, 1984).

Alternatively, any fusion protein can be readily purified by utilizing an antibody specific for the fusion protein being expressed. For example, one such system allows for the ready purification of non-denatured fusion proteins expressed in human cell lines (Janknecht et al., Proc. Natl. Acad. Sci. USA 88:8972-8976, 1991). In this system, the gene of interest is subcloned into a vaccinia recombination plasmid such that the open reading frame is translationally fused to an amino-terminal tag consisting of six histidine residues. Extracts from cells infected with recombinant vaccinia virus are loaded onto Ni²⁺-nitriloacetic acid-agarose columns, and histidine-tagged proteins are selectively eluted with imidazole-containing buffers.

Also encompassed by the present invention are fusion proteins that direct a membrane protein to a target organ and/or facilitate transport across the membrane into the cytosol. Conjugation of membrane proteins to antibody molecules, or their Fab fragments, could be used to target cells bearing a particular epitope. Attaching an appropriate signal sequence to a membrane protein would also transport the membrane protein to a desired location within the cell. Alternatively, targeting of membrane proteins or their nucleic acid sequences might be achieved using liposomes or lipid complex based delivery systems. Such technologies are described in “Liposomes: A Practical Approach” (New, R. R. C., ed., IRL Press, New York, N.Y., 1990), and in U.S. Pat. Nos. 4,594,595, 5,459,127, 5,948,767 and 6,110,490.

Additionally embodied are protein constructs engineered in such a way that they facilitate transport of a membrane protein to a target site or desired organ, where the membrane protein crosses the cell membrane and/or the nucleus and exerts its functional activity. This goal may be achieved by coupling of a membrane protein to a cytokine or other ligand that provides targeting specificity, and/or to a protein transducing domain (see, e.g., U.S. Provisional Patent Application Ser. Nos. 60/111,701 and 60/056,713, for examples of such transducing sequences), to facilitate passage across cellular membranes, and/or can optionally be engineered to include one or more nuclear localization signal(s).

Additionally contemplated are oligopeptides that are modeled on an amino acid sequence first described in the Sequence Listing. Such oligopeptides are generally between about 10 to about 100 amino acids long, or between about 16 to about 80 amino acids long, or between about 20 to about 35 amino acids long, or any variation or combination of sizes represented therein that incorporate a contiguous region of sequence first disclosed in the Sequence Listing. Such oligopeptides can be of any length disclosed within the above ranges and can initiate at any amino acid position represented in the Sequence Listing.

The invention also contemplates “substantially isolated” or “substantially pure” proteins or polypeptides. By a “substantially isolated” or “substantially pure” protein or polypeptide is meant a protein or polypeptide that has been separated from at least some of those components which naturally accompany it. Typically, the protein or polypeptide is substantially isolated or pure when it is at least 60%, by weight, free from the proteins and other naturally-occurring organic molecules with which it is naturally associated in vivo. Preferably, the purity of the preparation is at least 75%, more preferably at least 90%, and most preferably at least 99%, by weight. A substantially isolated or pure protein or polypeptide may be obtained, for example, by extraction from a natural source, by expression of a recombinant nucleic acid encoding the protein or polypeptide, or by chemically synthesizing the protein or polypeptide.

Purity can be measured by any appropriate method, e.g., column chromatography such as immunoaffinity chromatography using an antibody specific for the protein or polypeptide, polyacrylamide gel electrophoresis, or HPLC analysis. A protein or polypeptide is substantially free of naturally associated components when it is separated from at least some of those contaminants which accompany it in its natural state. Thus, a polypeptide which is chemically synthesized or produced in a cellular system different from the cell from which it naturally originates will be, by definition, substantially free from its naturally associated components. Accordingly, substantially isolated or pure proteins or polypeptides include eukaryotic proteins synthesized in E. coli, other prokaryotes, or any other organism in which they do not naturally occur.

### 7.3 Antibodies to Membrane Proteins

Antibodies that specifically recognize one or more epitopes of the disclosed membrane proteins, conserved variants of the disclosed membrane proteins, or peptide fragments of the disclosed membrane proteins, are also encompassed by the present invention. Such antibodies include, but are not limited to, polyclonal antibodies, monoclonal antibodies (mAbs), humanized or chimeric antibodies, single chain antibodies, Fab fragments, F(ab′)₂ fragments, fragments produced by a Fab expression library, anti-idiotypic (anti-Id) antibodies, and epitope-binding fragments of any of the above.

The antibodies of the invention may be used, for example, in the detection of the disclosed membrane proteins in a biological sample and may, therefore, be utilized as part of a diagnostic or prognostic technique whereby patients may be tested for abnormal amounts of one or more of the disclosed membrane proteins. Such antibodies may also be utilized in conjunction with, for example, compound screening schemes, as described herein, for the evaluation of the effect of test compounds on expression and/or activity of the disclosed membrane proteins. Additionally, such antibodies can be used in conjunction with gene therapy to, for example, evaluate the normal and/or engineered membrane protein-expressing cells prior to their introduction into a patient. Such antibodies may additionally be used as a method for the inhibition of abnormal membrane protein activity. Thus, such antibodies may be utilized as part of a variety of therapeutic regimens, such as, for example, in kidney disorder, digestive disorder, infertility, improper blood pressure, and/or body weight disorder treatment methods.

For the production of antibodies, various host animals may be immunized by injection with a disclosed membrane protein, one or more peptide from a membrane protein (e.g., one corresponding to a functional domain of the protein, such as an ECD, TM or CD), truncated membrane protein polypeptide(s) (membrane proteins in which one or more domains, e.g., a TM or CD, has been deleted), functional equivalents of a membrane protein, or mutant versions of a membrane protein. Such host animals may include, but are not limited to, rabbits, mice, and rats, to name but a few.

Various adjuvants may be used to increase the immunological response, depending on the host species, including, but not limited to, Freund's adjuvant (complete and incomplete), mineral salts such as aluminum hydroxide or aluminum phosphate, chitosan, surface active substances such as lysolecithin, pluronic polyols, polyanions, peptides, oil emulsions, and potentially useful human adjuvants such as BCG (bacille Calmette-Guerin) and Corynebacterium parvum. Alternatively, the immune response could be enhanced by combination and/or coupling with molecules such as keyhole limpet hemocyanin, tetanus toxoid, diphtheria toxoid, ovalbumin, cholera toxin, or fragments thereof.

Polyclonal antibodies are heterogeneous populations of antibody molecules derived from the sera of the immunized animals. Monoclonal antibodies, which are homogeneous populations of antibodies to a particular antigen, may be obtained by any technique that provides for the production of antibody molecules by continuous cell lines in culture. These include, but are not limited to, the hybridoma technique (Kohler and Milstein, Nature 256:495-497, 1975, and U.S. Pat. No. 4,376,110), the human B-cell hybridoma technique (Kosbor et al., Immunology Today 4:72, 1983, and Cole et al., Proc. Natl. Acad. Sci. USA 80:2026-2030, 1983), and the EBV-hybridoma technique (Cole et al., in “Monoclonal Antibodies and Cancer Therapy”, Vol. 27, UCLA Symposia on Molecular and Cellular Biology, New Series, pp. 77-96 (Reisfeld and Sell, eds., Alan R. Liss, Inc. New York, N.Y., 1985)). Such antibodies may be of any immunoglobulin class, including IgG, IgM, IgE, IgA, IgD, and any subclass thereof. The hybridomas producing the mAbs of this invention may be cultivated in vitro or in vivo. Production of high titers of mAbs in vivo makes this the presently preferred method of production.

In addition, techniques developed for the production of “chimeric antibodies” (Morrison et al., Proc. Natl. Acad. Sci. USA 81:6851-6855, 1984, Neuberger et al., Nature 312:604-608, 1984, and Takeda et al., Nature 314:452-454, 1985), by splicing the genes from a mouse antibody molecule of appropriate antigen specificity together with genes from a human antibody molecule of appropriate biological activity, can be used. A chimeric antibody is a molecule in which different portions are derived from different animal species, such as those having a variable region derived from a murine mAb and a human immunoglobulin constant region. Such technologies are described in U.S. Pat. Nos. 6,075,181 and 5,877,397. Also encompassed by the present invention is the use of fully humanized monoclonal antibodies, as described in U.S. Pat. No. 6,150,584.

Alternatively, techniques described for the production of single chain antibodies (U.S. Pat. No. 4,946,778, Bird, Science 242:423-426, 1988, Huston et al., Proc. Natl. Acad. Sci. USA 85:5879-5883, 1988, and Ward et al., Nature 341:544-546, 1989) can be adapted to produce single chain antibodies against membrane proteins, polypeptides, and/or peptides. Single chain antibodies are formed by linking the heavy and light chain fragments of the Fv region via an amino acid bridge, resulting in a single chain polypeptide.

Antibody fragments that recognize specific epitopes may be generated by known techniques. For example, such fragments include, but are not limited to: F(ab′)₂ fragments, which can be produced by pepsin digestion of an antibody molecule; and Fab fragments, which can be generated by reducing the disulfide bridges of F(ab′)₂ fragments. Alternatively, Fab expression libraries may be constructed (Huse et al., Science 246:1275-1281, 1989), to allow rapid and easy identification of monoclonal Fab fragments with the desired specificity.

Antibodies to the disclosed membrane proteins can, in turn, be utilized to generate anti-idiotype antibodies that “mimic” a given membrane protein, using techniques well-known to those skilled in the art (see, e.g., Greenspan and Bona, FASEB J. 7:437-444, 1993, and Nissinoff, J. Immunol. 147:2429-2438, 1991). For example antibodies that bind to an ECD of a membrane protein and competitively inhibit the binding of a ligand can be used to generate anti-idiotypes that “mimic” the ECD and, therefore, bind and neutralize the ligand. Such neutralizing anti-idiotypes, or Fab fragments of such anti-idiotypes, can be used in therapeutic regimens involving the membrane protein signaling pathway.

Additionally, given the high degree of relatedness of the disclosed membrane proteins across mammalian species, the presently described knock-out mice (having never seen the corresponding homologous membrane protein, and thus never been tolerized to the corresponding homologous membrane protein), have a unique utility, as they can be advantageously applied to the generation of antibodies against the disclosed mammalian membrane proteins (i.e., any of the disclosed membrane proteins will be immunogenic in the corresponding knock-out animals).

### 7.4 Diagnosis of Abnormalities Related to the Disclosed Proteins

A variety of methods can be employed for the diagnostic and prognostic evaluation of disorders related to the function of the disclosed membrane proteins, and for the identification of subjects having a predisposition to such disorders.

Such methods can, for example, utilize reagents such as the membrane protein nucleotide sequences described in Section 7.1, and/or membrane protein antibodies as described in Section 7.3. Specifically, such reagents may be used, for example, for: (1) the detection of the presence of membrane protein gene mutations, or the detection of either over- or under-expression of membrane protein mRNA relative to a given (i.e., normal) phenotype; (2) the detection of either an over- or an under-abundance of a membrane protein relative to a given (i.e., normal) phenotype; and (3) the detection of perturbations or abnormalities in a signal transduction pathway mediated by a membrane protein.

The methods described herein may be performed, for example, by utilizing pre-packaged diagnostic kits comprising at least one specific membrane protein nucleotide sequence and/or membrane protein antibody reagent described herein, which may be conveniently used, e.g., in clinical settings to diagnose patients exhibiting medical disorders or abnormalities, such as, for example, kidney disorders, digestive disorders, infertility, improper blood pressure, and/or body weight disorders.

For the detection of membrane protein mutations, any nucleated cell can be used as a starting source for genomic nucleic acid. For the detection of membrane protein gene expression or gene products, any cell-type or tissue in which a membrane protein gene is expressed can be utilized.

Nucleic acid-based detection techniques and peptide detection techniques are described in greater detail below.

### 7.4.1 Detection of Membrane Protein Genes and Transcripts

Mutations within a membrane protein gene or nucleotide sequence can be detected by utilizing a number of techniques. Nucleic acids from any nucleated cell can be used as the starting point for such assay techniques, and may be isolated according to standard nucleic acid preparation procedures that are well-known to those of skill in the art.

DNA may be used in hybridization or amplification assays of biological samples to detect abnormalities involving membrane protein gene structure, including point mutations, insertions, deletions and chromosomal rearrangements. Such assays may include, but are not limited to, Southern analyses, single stranded conformational polymorphism analyses, RFLP analyses (as generally described in U.S. Pat. No. 5,272,057), coding single nucleotide polymorphism analyses, and PCR analyses.

Such diagnostic methods for the detection of membrane protein gene-specific mutations can involve, for example, contacting and incubating nucleic acids, including recombinant DNA molecules, cloned genes, or degenerate variants thereof, obtained from a sample, e.g., derived from a patient sample or other appropriate cellular source, with one or more labeled nucleic acid reagents, including recombinant DNA molecules, cloned genes or degenerate variants thereof, as described in Section 7.1, under conditions favorable for the specific annealing of these reagents to their complementary sequences within a given membrane protein gene or sequence. Preferably, the lengths of these nucleic acid reagents are at least 15 to 30 nucleotides. After incubation, all non-annealed nucleic acids are removed from the nucleic acid:membrane protein molecule hybrid. The presence of nucleic acids that have hybridized, if any such molecules exist, is then detected. In conjunction with such a detection scheme, the nucleic acid from the cell-type or tissue of interest can be immobilized, for example, to a solid support such as a membrane, or a plastic surface such as that on a microtiter plate or polystyrene beads. In such a case, after incubation, non-annealed, labeled nucleic acid reagents of the type described in Section 7.1 are easily removed. Detection of the remaining, annealed, labeled membrane protein nucleic acid reagents is accomplished using standard techniques well-known to those in the art. The membrane protein sequences to which the nucleic acid reagents have annealed can be compared to the annealing pattern expected from a normal membrane protein sequence in order to determine whether a membrane protein mutation is present.

Alternative diagnostic methods for the detection of membrane protein gene specific nucleic acid molecules, in patient samples or other appropriate cell sources, may involve their amplification, e.g., by PCR (see, e.g., U.S. Pat. Nos. 4,683,195; 4,683,202 and 4,800,159), followed by the detection of the amplified molecules using techniques well-known to those of skill in the art. The resulting amplified sequences can be compared to those that would be expected if the nucleic acid being amplified contained only normal copies of a membrane protein gene in order to determine whether a membrane protein gene mutation exists.

Well-known genotyping techniques can also be performed to identify individuals carrying membrane protein gene mutations. Such techniques include, for example, the use of RFLPs, which involve sequence variations in one of the recognition sites for the specific restriction enzyme used. Additionally, improved methods for analyzing DNA polymorphisms that can be utilized for the identification of membrane protein gene mutations have been described, which capitalize on the presence of variable numbers of short, tandemly repeated DNA sequences between restriction enzyme sites. For example, U.S. Pat. No. 5,075,217 describes a DNA marker based on length polymorphisms in blocks of (dC-dA)ₙ-(dG-dT)ₙ short tandem repeats. The average separation of (dC-dA)ₙ-(dG-dT)ₙ blocks is estimated to be 30,000-60,000 bp. Markers that are so closely spaced exhibit a high frequency co-inheritance, and are extremely useful in the identification of genetic mutations, such as, for example, mutations within a given membrane protein gene, and the diagnosis of diseases and disorders related to membrane protein mutations. U.S. Pat. No. 5,364,759 describes a DNA profiling assay for detecting short tri- and tetra-nucleotide repeat sequences. The process includes extracting the DNA of interest, such as the membrane protein gene, amplifying the extracted DNA, and labeling the repeat sequences to form a genotypic map of the individual's DNA.

The level of membrane protein gene expression can also be assayed by detecting and measuring transcription levels. For example, RNA from a cell-type or tissue known to express, or suspected of expressing, a membrane protein gene can be isolated and tested utilizing hybridization or PCR techniques such as those described herein. The isolated cells can be derived from cell culture or from a patient. The analysis of cells taken from culture may be a necessary step in the assessment of cells to be used as part of a cell-based gene therapy technique or, alternatively, to test the effect of compounds on the expression of a membrane protein gene. Such analyses may reveal both quantitative and qualitative aspects of the expression pattern of a membrane protein gene, including activation or inactivation of membrane protein gene expression.

In one embodiment of such a detection scheme, cDNAs are synthesized from the RNAs of interest (e.g., by reverse transcription of the RNA molecule into cDNA). A sequence within the cDNA is then used as the template for a nucleic acid amplification reaction, such as a PCR amplification reaction, or the like. The nucleic acid reagents used as synthesis initiation reagents (e.g., primers) in the reverse transcription and nucleic acid amplification steps of this method are chosen from among the membrane protein nucleic acid reagents described in Section 7.1. The preferred lengths of such nucleic acid reagents are at least 9-30 nucleotides. For detection of the amplified product, the nucleic acid amplification may be performed using radioactively or non-radioactively labeled nucleotides. Alternatively, enough amplified product may be made such that the product may be visualized by standard ethidium bromide staining or any other suitable nucleic acid staining method, or by sequencing.

It is also possible to perform such membrane protein gene expression assays “in situ”, i.e., directly upon tissue sections (fixed and/or frozen) of patient tissue obtained from biopsies or resections, such that no nucleic acid purification is necessary. Nucleic acid reagents, such as those described herein, may be used as probes and/or primers for such in situ procedures (see, e.g., “PCR In Situ Hybridization: Protocols And Applications” (Nuovo, ed., Raven Press, New York, N.Y., 1992)). Alternatively, if a sufficient quantity of the appropriate cells can be obtained, standard Northern analysis can be performed to determine the level of membrane protein mRNA expression.

Additionally, membrane protein oligonucleotide or polynucleotide sequences can be used as hybridization probes in conjunction with a solid support matrix/substrate (e.g., resins, beads, membranes, plastics, polymers, metal or metallized substrates, gene chips, and crystalline or polycrystalline substrates, etc.).

### 7.4.2 Detection of Membrane Protein Products

Antibodies directed against wild-type or mutant membrane proteins, or conserved variants or peptide fragments thereof, which are discussed above, may also be used as diagnostics and prognostics, as described herein. Such diagnostic methods may be used to detect abnormalities in the level of membrane protein gene expression, or abnormalities in the structure and/or temporal, tissue, cellular, or subcellular location of a membrane protein, and may be performed in vivo or in vitro, such as, for example, on biopsy tissue.

For example, antibodies directed to epitopes of an ECD of a membrane protein can be used in vivo to detect the pattern and level of expression of the particular membrane protein in the body. Such antibodies can be labeled, e.g., with a radio-opaque or other appropriate compound, and injected into a subject in order to visualize binding to membrane proteins in the body, using methods such as X-rays, CAT-scans, or MRI. Labeled antibody fragments, e.g., the Fab or single chain antibody comprising the smallest portion of the antigen binding region, are preferred for this purpose, in order to promote crossing the blood-brain barrier and permit labeling of membrane proteins expressed in the brain.

Additionally, any membrane protein fusion or conjugated protein whose presence can be detected can be administered. For example, membrane protein fusion or conjugated proteins labeled with a radio-opaque or other appropriate compound can be administered and visualized in vivo, as discussed above for labeled antibodies. Further, fusion proteins such as alkaline phosphatase-membrane protein or membrane protein-alkaline phosphatase can be utilized for in vitro diagnostic procedures.

Alternatively, immunoassays or fusion protein detection assays, as described herein, can be utilized on biopsy and autopsy samples in vitro to permit assessment of the expression pattern of a membrane protein. Such assays are not confined to the use of antibodies that define an ECD of a membrane protein, but can include the use of antibodies directed to epitopes of any of the domains of a membrane protein, e.g., the ECD, the TM and/or CD. The use of each or all of these labeled antibodies will yield useful information regarding translation and intracellular transport of a membrane protein to the cell surface, and can identify defects in processing.

The tissue or cell-type to be analyzed will generally include those that are known to express, or suspected of expressing, a membrane protein gene. The protein isolation methods employed herein may, for example, be such as those previously described (“Antibodies: A Laboratory Manual”, supra). The isolated cells can be derived from cell culture or a patient. The analysis of cells taken from culture may be a necessary step in the assessment of cells that could be used as part of a cell-based gene therapy technique or, alternatively, to test the effect of compounds on the expression of a membrane protein gene.

For example, antibodies, or fragments of antibodies, such as those described in Section 7.3, may be used to quantitatively or qualitatively detect the presence of membrane proteins, or conserved variants or peptide fragments thereof. This can be accomplished, for example, by immunofluorescence techniques employing a fluorescently labeled antibody (see below, this Section), coupled with light microscopic, flow cytometric, or fluorimetric detection. Such techniques are especially preferred if such membrane proteins are expressed on the cell surface.

The antibodies (or fragments thereof), or membrane protein fusion or conjugated proteins, may additionally be employed histologically, as in immunofluorescence, immunoelectron microscopy or non-immuno assays, for in situ detection of membrane proteins, or conserved variants or peptide fragments thereof, or for membrane protein binding (in the case of labeled membrane protein ligand fusion proteins).

In situ detection may be accomplished by removing a histological specimen from a patient, and applying thereto a labeled antibody or fusion protein of the present invention. The antibody (or fragment) or fusion protein is preferably applied by overlaying the labeled moiety onto a biological sample. Through the use of such a procedure, it is possible to determine not only the presence of a membrane protein, or conserved variants or peptide fragments, or membrane protein binding, but also membrane protein distribution in the examined tissue. Using the present invention, those of ordinary skill will readily perceive that any of a wide variety of histological methods (such as staining procedures) can be modified in order to achieve such in situ detection.

Immunoassays and non-immunoassays for membrane proteins, or conserved variants or peptide fragments thereof, will typically comprise incubating a sample, such as a biological fluid, a tissue extract, freshly harvested cells, or lysates of cells that have been incubated in cell culture, in the presence of a detectably labeled antibody or fusion protein capable of identifying a membrane protein, or conserved variants or peptide fragments thereof, and detecting the bound antibody or fusion protein by any of a number of techniques well-known in the art.

The biological sample may be brought in contact with and immobilized onto a solid phase support or carrier, such as nitrocellulose or any other solid support that is capable of immobilizing cells, cell particles, or soluble proteins. The support may then be washed with suitable buffers, followed by treatment with the detectably labeled membrane protein antibody or membrane protein ligand fusion protein. The solid phase support may then be washed with the buffer a second time to remove unbound antibody or fusion protein. The amount of bound label remaining on the solid support may then be detected by conventional means.

By “solid phase support or carrier” is intended any support capable of binding an antigen or an antibody. Well-known supports or carriers include, but are not limited to, glass, polystyrene, polypropylene, polyethylene, dextran, nylon, amylases, natural and modified celluloses, polyacrylamides, gabbros, and magnetite. The nature of the carrier can be either soluble to some extent or insoluble for the purposes of the present invention. The support material can have virtually any possible structural configuration so long as the coupled molecule is capable of binding to an antigen or antibody. Thus, the support configuration may be spherical, as in a bead, or cylindrical, as in the inside surface of a test tube, or the external surface of a rod. Alternatively, the surface may be flat, such as a sheet or test strip. Preferred supports include polystyrene beads. Those skilled in the art will know many other suitable carriers for binding an antibody or antigen, or will be able to ascertain the same by use of routine experimentation.

The binding activity of a given lot of membrane protein antibody or membrane protein ligand fusion protein may be determined according to well-known methods. Those skilled in the art will be able to determine operative and optimal assay conditions for each determination by employing routine experimentation.

With respect to antibodies, one of the ways in which a membrane protein antibody can be detectably labeled is by linking it to an enzyme for use in an enzyme immunoassay (Voller, Diagnostic Horizons 2:1-7, 1978; Voller et al., J. Clin. Pathol. 31:507-520, 1978; Butler, Meth. Enzymol. 73:482-523, 1981; 1980, “Enzyme Immunoassay” (Maggio, E., ed., CRC Press, Boca Raton, Fla., 1980); and “Enzyme Immunoassay” (Ishikawa et al., eds., Igaku-Shoin, Tokyo, Japan, 1981)). The enzyme that is bound to the antibody will react with an appropriate substrate, preferably a chromogenic substrate, in such a manner as to produce a chemical moiety that can be detected, for example, by spectrophotometric, fluorimetric, or visual means. Enzymes that can be used to detectably label the antibody include, but are not limited to, malate dehydrogenase, staphylococcal nuclease, delta-5-steroid isomerase, yeast alcohol dehydrogenase, alpha-glycerophosphate, dehydrogenase, triose phosphate isomerase, horseradish peroxidase, alkaline phosphatase, asparaginase, glucose oxidase, beta-galactosidase, ribonuclease, urease, catalase, glucose-6-phosphate dehydrogenase, glucoamylase and acetylcholinesterase. The detection can be accomplished by calorimetric methods, which employ a chromogenic substrate for the enzyme. Detection may also be accomplished by visual comparison of the extent of enzymatic reaction of a substrate in comparison with similarly prepared standards.

Detection may also be accomplished using any of a variety of other immunoassays. For example, by radioactively labeling the antibodies or antibody fragments it is possible to detect a membrane protein through the use of a radioimmunoassay (see, e.g., “Principles of Radioimmunoassays” (Weintraub, B., ed., Seventh Training Course on Radioligand Assay Techniques, The Endocrine Society, Chevy Chase, Md., March, 1986)). The radioactive isotope can be detected by such means as the use of a gamma counter, a scintillation counter, or by autoradiography.

It is also possible to label the antibody with a fluorescent compound. When the fluorescently labeled antibody is exposed to light of the proper wavelength, its presence can be detected due to fluorescence. Among the most commonly used fluorescent labeling compounds are fluorescein isothiocyanate, rhodamine, phycoerythrin, phycocyanin, allophycocyanin, o-phthaldehyde and fluorescamine. The antibody can also be detectably labeled using fluorescence emitting metals such as ¹⁵²Eu, or others of the lanthanide series. These metals can be attached to the antibody using such metal chelating groups as diethylenetriaminepentacetic acid or ethylenediaminetetraacetic acid.

The antibody also can be detectably labeled by coupling it to a chemiluminescent compound. The presence of the chemiluminescent-tagged antibody is then determined by detecting the presence of luminescence that arises during the course of a chemical reaction. Examples of particularly useful chemiluminescent labeling compounds are luminol, isoluminol, theromatic acridinium ester, imidazole, acridinium salt and oxalate ester.

Likewise, a bioluminescent compound may be used to label the antibodies (or fragments thereof) of the present invention. Bioluminescence is a type of chemiluminescence found in biological systems, in which a catalytic protein increases the efficiency of the chemiluminescent reaction. The presence of a bioluminescent protein is determined by detecting the presence of luminescence. Important bioluminescent compounds for purposes of labeling are luciferin, luciferase, and aequorin (green fluorescent protein and mutants thereof, as described in U.S. Pat. Nos. 5,491,084, 5,625,048, 5,777,079, 5,795,737, 5,804,387, 5,874,304, 5,968,750, 5,976,796, 6,020,192, 6,027,881, 6,054,321, 6,096,865, 6,146,826, 6,172,188 and 6,265,548).

### 7.5 Screening Assays for Compounds That Modulate Membrane Protein Expression or Activity

The following assays are designed to identify: compounds that interact with (e.g., bind to) membrane proteins (including, but not limited to, an ECD or CD of a membrane protein); intracellular proteins that interact with a membrane protein (including, but not limited to, a TM or CD of a membrane protein); compounds that interfere with the interaction of a membrane protein with transmembrane or intracellular proteins involved in membrane protein-mediated signal transduction; and compounds that modulate the activity of a membrane protein gene (i.e., modulate the level of membrane protein gene expression) or modulate the level of a membrane protein. Assays may additionally be utilized that identify compounds that bind to membrane protein gene regulatory sequences (e.g., promoter sequences), and may modulate membrane protein gene expression (see, e.g., Platt et al., J. Biol. Chem. 269:28558-28562, 1994).

The compounds that can be screened in accordance with the invention include, but are not limited to, peptides, antibodies and fragments thereof, and other organic compounds (e.g., peptidomimetics) that bind to an ECD of a membrane protein, and either mimic the activity triggered by the natural ligand (i.e., agonists) or inhibit the activity triggered by the natural ligand (i.e., antagonists); as well as peptides, antibodies or fragments thereof, and other organic compounds that mimic the ECD of a membrane protein (or a portion thereof) and bind to and “neutralize” a natural ligand of the membrane protein.

Such compounds may include, but are not limited to, peptides, such as, for example, soluble peptides, including, but not limited to, members of random peptide libraries (see, e.g., Lam et al., Nature 354:82-84, 1991, and Houghten et al., Nature 354:84-86, 1991), and combinatorial chemistry-derived molecular libraries made of D- and/or L-configuration amino acids, phosphopeptides (including, but not limited to, members of random or partially degenerate, directed phosphopeptide libraries; see, e.g., Songyang et al., Cell 72:767-778, 1993), antibodies (including, but not limited to, polyclonal, monoclonal, humanized, anti-idiotypic, chimeric, or single chain antibodies, and Fab, F(ab′)₂ and Fab expression library fragments, and epitope-binding fragments thereof), and small organic or inorganic molecules.

Other compounds that can be screened in accordance with the invention include, but are not limited to, small organic molecules that are able to cross the blood-brain barrier, gain entry into an appropriate cell (e.g., in the cerebellum, the hypothalamus, etc.), and affect the expression of a membrane protein gene or some other gene involved in the membrane protein signal transduction pathway (e.g., by interacting with the regulatory region or transcription factors involved in gene expression); or such compounds that affect the activity of a membrane protein (e.g., by inhibiting or enhancing the enzymatic activity of a CD) or the activity of some other intracellular factor involved in the membrane protein signal transduction pathway.

Computer modeling and searching technologies permit identification of compounds, or the improvement of already identified compounds, that can modulate membrane protein expression or activity. Having identified such a compound or composition, the active sites or regions are identified. Such active sites might typically be ligand binding sites. The active site can be identified using methods known in the art including, for example, from the amino acid sequences of peptides, from the nucleotide sequences of nucleic acids, or from study of complexes of the relevant compound or composition with its natural ligand. In the latter case, chemical or X-ray crystallographic methods can be used to find the active site by finding where on the factor the complexed ligand is found.

Next, the three dimensional geometric structure of the active site is determined. This can be done by known methods, including X-ray crystallography, which can determine a complete molecular structure. On the other hand, solid or liquid phase NMR can be used to determine certain intra-molecular distances. Any other experimental method of structure determination can be used to obtain partial or complete geometric structures. The geometric structures may be measured with a complexed ligand, natural or artificial, which may increase the accuracy of the active site structure determined.

If an incomplete or insufficiently accurate structure is determined, the methods of computer based numerical modeling can be used to complete the structure or improve its accuracy. Any recognized modeling method may be used, including parameterized models specific to particular biopolymers such as proteins or nucleic acids, molecular dynamics models based on computing molecular motions, statistical mechanics models based on thermal ensembles, or combined models. For most types of models, standard molecular force fields, representing the forces between constituent atoms and groups, are necessary, and can be selected from force fields known in physical chemistry. The incomplete or less accurate experimental structures can serve as constraints on the complete and more accurate structures computed by such modeling methods.

Finally, having determined the structure of the active site, either experimentally, by modeling, or by a combination thereof, candidate modulating compounds can be identified by searching databases containing compounds along with information on their molecular structure. Such a search seeks compounds having structures that match the determined active site structure and that interact with the groups defining the active site. Such a search can be manual, but is preferably computer assisted. The compounds found from such a search are potential membrane protein modulating compounds.

Alternatively, these methods can be used to identify improved modulating compounds from an already known modulating compound or ligand. The composition of the known compound can be modified, and the structural effects of modification can be determined using the experimental and computer modeling methods described above applied to the new composition. The altered structure is then compared to the active site structure of the compound to determine if an improved fit or interaction results. In this manner systematic variations in composition, such as by varying side groups, can be quickly evaluated to obtain modified modulating compounds or ligands of improved specificity or activity. Further experimental and computer modeling methods useful to identify modulating compounds based upon identification of the active sites of a membrane protein, and related transduction and transcription factors, will be apparent to those of skill in the art.

Examples of molecular modeling systems are the CHARMM and QUANTA programs (Polygen Corporation, Waltham, Mass.). CHARMM performs the energy minimization and molecular dynamics functions, while QUANTA performs the construction, graphic modeling and analysis of molecular structure. QUANTA allows interactive construction, modification, visualization, and analysis of the behavior of molecules with each other.

A number of articles review computer modeling of drugs interactive with specific proteins, such as: Rotivinen et al., Acta Pharmaceutical Fennica 97:159-166, 1988, Ripka, New Scientist, pp. 54-57, June 16, 1988, McKinaly and Rossmann, Ann. Rev. Pharmacol. Toxiciol. 29:111-122, 1989, Perry and Davies, in “QSAR: Quantitative Structure-Activity Relationships in Drug Design”, pp. 189-193 (Fauchere, ed., Alan R. Liss, Inc., New York, N.Y., 1989), Lewis and Dean, Proc. R. Soc. Lond. 236:125-140 and 141-162, 1989; and, with respect to a model receptor for nucleic acid components, Askew et al., J. Am. Chem. Soc. 111:1082-1090, 1989. Other computer programs that screen and graphically depict chemicals are available from companies such as BioDesign, Inc. (Pasadena, Calif.), Allelix, Inc. (Mississauga, Ontario, Canada), and Hypercube, Inc. (Cambridge, Ontario). Although these are primarily designed for application to drugs specific to particular proteins, they can be adapted to the design of drugs specific to membrane proteins, or regions of DNA or RNA, once that region is identified.

Although described above with reference to design and generation of compounds that could alter binding, one could also screen libraries of known compounds, including natural products or synthetic chemicals, and biologically active materials, including proteins, for compounds that are inhibitors or activators.

Cell-based systems can also be used to identify compounds that bind membrane proteins, as well as assess the altered activity associated with such binding in living cells. One tool of particular interest for such assays is green fluorescent protein, which is described, inter alia, in U.S. Pat. No. 5,625,048. Cells that may be used in such cellular assays include, but are not limited to, leukocytes, or cell lines derived from leukocytes, lymphocytes, stem cells, including embryonic stem cells, and the like. In addition, expression host cells (e.g., B95 cells, COS cells, CHO cells, OMK cells, fibroblasts, Sf9 cells) genetically engineered to express a functional membrane protein of interest and to respond to activation by the test, or natural, ligand, as measured by a chemical or phenotypic change, or induction of another host cell gene, can be used as an end point in the assay.

### 7.5.1 In Vitro Screening Assays for Compounds That Bind to Membrane Proteins

In vitro systems can be designed to identify compounds capable of interacting with (e.g., binding to) a membrane protein (including, but not limited to, an ECD or CD of a membrane protein). Compounds identified may be useful, for example, in modulating the activity of wild-type and/or mutant membrane proteins, in elaborating a biological function of a membrane protein, in screens for identifying compounds that disrupt normal membrane protein interactions, or may in themselves disrupt such interactions.

The principle of the assays used to identify compounds that bind to a membrane protein involves preparing a reaction mixture of a membrane protein and a test compound under conditions and for a time sufficient to allow the two components to interact and bind, thus forming a complex that can be removed and/or detected in the reaction mixture. The membrane protein species used can vary depending upon the goal of the screening assay. For example, where agonists of the natural ligand are sought, the full length membrane protein, a soluble truncated membrane protein, e.g., in which the TM and/or CD is deleted from the molecule, a peptide corresponding to an ECD, or a fusion protein containing one or more ECD fused to a protein or polypeptide that affords advantages in the assay system (e.g., labeling, isolation of the resulting complex, etc.), can be utilized. Where compounds that interact with the CD are sought to be identified, peptides corresponding to a membrane protein CD or fusion proteins containing a membrane protein CD can be used.

The screening assays can be conducted in a variety of ways. For example, one method to conduct such an assay would involve anchoring the membrane protein, polypeptide, peptide, or fusion protein, or the test substance, onto a solid phase and detecting membrane protein/test compound complexes anchored on the solid phase at the end of the reaction. In one embodiment of such a method, the membrane protein reactant may be anchored onto a solid surface, and the test compound, which is not anchored, may be labeled, either directly or indirectly. Examples of some of the technologies available to immobilize the molecules are discussed in “Immobilized Biomolecules In Analysis: A Practical Approach” (Cass and Ligler, eds., Oxford University Press, New York, N.Y., 1999).

In practice, microtiter plates may conveniently be utilized as the solid phase. The anchored component may be immobilized by non-covalent or covalent attachments. Non-covalent attachment may be accomplished by simply coating the solid surface with a solution of the protein and drying. Alternatively, an immobilized antibody, preferably a monoclonal antibody specific for the protein to be immobilized, may be used to anchor the protein to the solid surface. The surfaces may be prepared in advance and stored.

In order to conduct the assay, the non-immobilized component is added to the coated surface containing the anchored component. After the reaction is complete, unreacted components are removed (e.g., by washing) under conditions such that any complexes formed will remain immobilized on the solid surface. The detection of complexes anchored on the solid surface can be accomplished in a number of ways. Where the previously non-immobilized component is pre-labeled, the detection of label immobilized on the surface indicates that complexes were formed. Where the previously non-immobilized component is not pre-labeled, an indirect label can be used to detect complexes anchored on the surface, e.g., using a labeled antibody specific for the previously non-immobilized component (the antibody, in turn, may be directly labeled or indirectly labeled with a labeled anti-Ig antibody).

Alternatively, a reaction can be conducted in a liquid phase, the reaction products separated from unreacted components, and complexes detected, e.g., using an immobilized antibody specific for a membrane protein, polypeptide, peptide, or fusion protein, or the test compound, to anchor any complexes formed in solution, and a labeled antibody specific for the other component of the possible complex to detect anchored complexes.

Alternatively, cell-based assays can be used to identify compounds that interact with a membrane protein. To this end, cell lines that express a membrane protein, or cell lines (e.g., COS cells, CHO cells, fibroblasts, etc.) that have been genetically engineered to express a membrane protein (e.g., by transfection or transduction of membrane protein DNA sequence) can be used. Interaction of the test compound with, for example, an ECD of a membrane protein expressed by the host cell can be determined by comparison or competition with native ligand.

### 7.5.2 Assays for Intracellular Proteins That Interact With Membrane Proteins

Any method suitable for detecting protein-protein interactions may be employed for identifying transmembrane proteins or intracellular proteins that interact with a membrane protein. Among the traditional methods that may be employed are co-immunoprecipitation, crosslinking and co-purification through gradients or chromatographic columns of cell lysates, or proteins obtained from cell lysates, and a membrane protein to identify proteins in the lysate that interact with the membrane protein. For these assays, the membrane protein component used can be a full length membrane protein, a soluble derivative lacking the membrane-anchoring region (e.g., a truncated membrane protein in which a TM is deleted resulting in a truncated molecule containing an ECD fused to a CD), a peptide corresponding to a CD, or a fusion protein containing a CD of a membrane protein. Once isolated, such an intracellular protein can be identified and can, in turn, be used in conjunction with standard techniques to identify proteins with which it interacts. For example, at least a portion of the amino acid sequence of an intracellular protein that interacts with a membrane protein can be ascertained using techniques well-known to those of skill in the art, such as via the Edman degradation technique (see, e.g., “Proteins: Structures and Molecular Principles”, pp.34-49, supra). The amino acid sequence obtained may be used as a guide for the generation of oligonucleotide mixtures that can be used to screen for nucleotide sequences encoding such intracellular proteins. Screening can be accomplished, for example, by standard hybridization or PCR techniques. Techniques for the generation of oligonucleotide mixtures and the screening are well-known (see, e.g., “Current Protocols in Molecular Biology”, supra, and “PCR Protocols: A Guide to Methods and Applications” (Innis et al., eds., Academic Press, Inc., New York, N.Y., 1990)).

Additionally, methods may be employed that result in the simultaneous identification of genes that encode transmembrane or intracellular proteins that interact with a membrane protein of the present invention. These methods include, for example, probing expression libraries, in a manner similar to the well-known technique of antibody probing of λgt11 libraries, using a labeled membrane protein, polypeptide, peptide, or fusion protein, e.g., a membrane protein polypeptide or domain fused to a marker (e.g., an enzyme, fluor, luminescent protein, or dye), or an Ig-Fc domain.

One method that detects protein interactions in vivo, the two-hybrid system, is described in detail for illustration only, and not by way of limitation. One version of this system utilizes yeast cells (Chien et al., Proc. Natl. Acad. Sci. USA 88:9578-9582, 1991), while another uses mammalian cells (Luo et al., Biotechniques 22:350-352, 1997). Both the yeast and mammalian two-hybrid systems are commercially available from Clontech (Palo Alto, Calif.), and are further described in U.S. Pat. Nos. 5,283,173; 5,468,614, and 5,667,973.

Briefly, utilizing such a system, plasmids are constructed that encode two hybrid proteins: one plasmid consists of nucleotides encoding the DNA-binding domain of a transcription activator protein fused to a membrane protein nucleotide sequence encoding a membrane protein, polypeptide, peptide, or fusion protein, and the other plasmid consists of nucleotides encoding an activation domain of a transcription activator protein fused to a cDNA encoding an unknown protein to be tested for interaction with a membrane protein, which has been recombined into this plasmid as part of a cDNA library. The DNA-binding domain fusion plasmid and the cDNA library are transformed into a strain of the yeast Saccharomyces cerevisiae or a mammalian cell (such as Saos-2, CHO, CV1, Jurkat or HeLa) that contains a reporter gene (e.g., HBS, lacZ, CAT, or a gene encoding an essential amino acid) whose regulatory region contains the binding site of the transcription activator. Either hybrid protein alone cannot activate transcription of the reporter gene: the DNA-binding domain hybrid cannot because it does not provide activation function; and the activation domain hybrid cannot because it cannot localize to the binding site of the activator. Interaction of the two hybrid proteins reconstitutes the functional activator protein and results in expression of the reporter gene, which is detected by an assay for the reporter gene product.

The two-hybrid system, or related methodologies, may be used to screen activation domain libraries for proteins that interact with the “bait” gene product. By way of example, and not by way of limitation, a membrane protein of the present invention may be used as the bait gene product. Total genomic or CDNA sequences are fused to DNA encoding an activation domain. This library and a plasmid encoding a hybrid of a bait membrane protein moiety fused to the DNA-binding domain are co-transformed into a reporter strain, and the resulting transformants are screened for those that express the reporter gene. For example, and not by way of limitation, a bait membrane protein gene sequence, such as an open reading frame of a membrane protein (or a domain of a membrane protein), can be cloned into a vector such that it is translationally fused to DNA encoding the DNA-binding domain of the GAL4 protein. These colonies are purified and the library plasmids responsible for reporter gene expression are isolated. DNA sequencing is then used to identify the proteins encoded by the library plasmids.

A cDNA library of the cell line from which proteins that interact with bait membrane protein moiety are to be detected can be made using methods routinely practiced in the art. According to one particular system, for example, the cDNA fragments can be inserted into a vector such that they are translationally fused to the transcriptional activation domain of GAL4. This library can be co-transformed along with the bait membrane protein gene-GAL4 fusion plasmid into a yeast strain that: a) cannot grow without added histidine; and b) contains a HIS3 gene driven by a promoter that contains GAL4 activation sequence. A cDNA encoded protein, fused to a GAL4 transcriptional activation domain, which interacts with bait membrane protein moiety will reconstitute an active GAL4 protein, and thereby drive expression of the HIS3 gene. Colonies that express HIS3 can be detected by growth on petri dishes containing semi-solid agar based media lacking histidine. The cDNA can then be purified from these strains, and used to produce and isolate the bait membrane protein moiety-interacting protein using techniques routinely practiced in the art.

### 7.5.3 Assays for Compounds That Interfere With Membrane Protein/Intracellular or Transmembrane Macromolecule Interaction

Macromolecules that interact with a membrane protein of the present invention are referred to, for purposes of this discussion, as “binding partners”. These binding partners are likely to be involved in a membrane protein signal transduction pathway. Therefore, it is desirable to identify compounds that interfere with or disrupt the interaction of such binding partners with membrane proteins, which may be useful in regulating the activity of a membrane protein and controlling disorders associated with membrane protein activity. Based on the similarity data and expression patterns of the disclosed membrane proteins, they are contemplated to be particularly useful in methods for identifying compounds useful in the therapeutic treatment of, for example, high or low blood pressure, kidney disorders, weight control disorders (or as taste maskers), alcoholism, metabolic disorders, cancer, mood and/or motivation disorders, particularly those involving light cycles, such as seasonal affected disorder, and sleep disorders including, but not limited to, “jet lag”, eating or behavioral disorders, mental illness, paralysis or palsy, nerve damage or degeneration, vision disorders, pain, fever, inflammatory disorders, sepsis, pancreatitis, sexual disorders, such as erectile dysfunction, infertility, and impotence, disorders of the immune system and autoimmune diseases such as diabetes, arthritis, inflammatory bowel disorder, irritable bowel syndrome, Crohn's disease, and ulcerative colitis, osteoporosis, connective tissue disorders, atherosclerosis, heart disease, heart rate disorders, and any associated symptoms.

The basic principle of the assay systems used to identify compounds that interfere with the interaction between a membrane protein and its binding partner or partners involves preparing a reaction mixture containing a membrane protein, polypeptide, peptide, or fusion protein, as described in Sections 7.5.1 and 7.5.2, and the binding partner under conditions and for a time sufficient to allow the two components to interact and bind, thus forming a complex. In order to test a compound for inhibitory activity, the reaction mixture is prepared in the presence and absence of the test compound. The test compound may be initially included in the reaction mixture, or may be added at a time subsequent to the addition of the membrane protein moiety and its binding partner. Control reaction mixtures are incubated without the test compound or with a placebo. The formation of any complexes between the membrane protein moiety and the binding partner is then detected. The formation of a complex in the control reaction, but not in the reaction mixture containing the test compound, indicates that the compound interferes with the interaction of the membrane protein and the interactive binding partner. Additionally, complex formation within reaction mixtures containing the test compound and a normal membrane protein may also be compared to complex formation within reaction mixtures containing the test compound and a mutant membrane protein. This comparison may be important in those cases wherein it is desirable to identify compounds that specifically disrupt interactions of mutant, or mutated, membrane proteins, but not normal membrane proteins.

The assay for compounds that interfere with the interaction of a membrane protein and its binding partner(s) can be conducted in a heterogeneous or homogeneous format. Heterogeneous assays involve anchoring either the membrane protein moiety or the binding partner onto a solid phase and detecting complexes anchored on the solid phase at the end of the reaction. In homogeneous assays, the entire reaction is carried out in a liquid phase. In either approach, the order of addition of reactants can be varied to obtain different information about the compounds being tested. For example, test compounds that interfere with the interaction by competition can be identified by conducting the reaction in the presence of the test substance, i.e., by adding the test substance to the reaction mixture prior to, or simultaneously with, a membrane protein moiety and interactive binding partner. Alternatively, test compounds that disrupt preformed complexes, e.g., compounds with higher binding constants that displace one of the components from the complex, can be tested by adding the test compound to the reaction mixture after complexes have been formed. The various formats are described briefly below.

In a heterogeneous assay system, either a membrane protein moiety or an interactive binding partner is anchored onto a solid surface, while the non-anchored species is labeled, either directly or indirectly. In practice, microtiter plates are conveniently utilized. The anchored species may be immobilized by non-covalent or covalent attachments. Non-covalent attachment may be accomplished simply by coating the solid surface with a solution of a membrane protein moiety or binding partner and drying. Alternatively, an immobilized antibody specific for the species to be anchored may be used to anchor the species to the solid surface. The surfaces may be prepared in advance and stored.

In order to conduct the assay, the partner of the immobilized species is exposed to the coated surface with or without the test compound. After the reaction is complete, unreacted components are removed (e.g., by washing) and any complexes formed will remain immobilized on the solid surface. The detection of complexes anchored on the solid surface can be accomplished in a number of ways. Where the non-immobilized species is pre-labeled, the detection of label immobilized on the surface indicates that complexes were formed. Where the non-immobilized species is not pre-labeled, an indirect label can be used to detect complexes anchored on the surface, e.g., using a labeled antibody specific for the initially non-immobilized species (the antibody, in turn, may be directly labeled or indirectly labeled with a labeled anti-Ig antibody). Depending upon the order of addition of reaction components, test compounds that inhibit complex formation or that disrupt preformed complexes can be detected.

Alternatively, the reaction can be conducted in a liquid phase in the presence or absence of the test compound, the reaction products separated from unreacted components, and complexes detected, e.g., using an immobilized antibody specific for one of the binding components to anchor any complexes formed in solution, and a labeled antibody specific for the other partner to detect anchored complexes. Again, depending upon the order of addition of reactants to the liquid phase, test compounds that inhibit complex or that disrupt preformed complexes can be identified.

In an alternate embodiment of the invention, a homogeneous assay can be used. In this approach, a preformed complex of a membrane protein moiety and an interactive binding partner is prepared in which either the membrane protein moiety or its binding partner is labeled, but the signal generated by the label is quenched due to formation of the complex (see, e.g., U.S. Pat. No. 4,109,496, which utilizes this approach for immunoassays). The addition of a test substance that competes with and displaces one of the species from the preformed complex will result in the generation of a signal above background. In this way, test substances that disrupt interactions between membrane proteins and intracellular binding partners can be identified.

In a particular embodiment, a membrane protein fusion protein can be prepared for immobilization. For example, a membrane protein (or peptide fragment, e.g., corresponding to a CD) can be fused to a glutathione-S-transferase (GST) gene using a fusion vector, such as pGEX-5X-1, in such a manner that its binding activity is maintained in the resulting fusion protein. The interactive binding partner can be purified and used to raise a monoclonal antibody, using methods routinely practiced in the art and described in Section 7.3. This antibody can be labeled with a radioactive isotope, ¹²⁵I for example, by methods routinely practiced in the art. In a heterogeneous assay, e.g., the GST-membrane protein fusion protein can be anchored to glutathione-agarose beads. The interactive binding partner can then be added in the presence or absence of the test compound in a manner that allows interaction and binding to occur. At the end of the reaction period, unbound material can be washed away, and the labeled monoclonal antibody can be added to the system and allowed to bind to the complexed components. The interaction between a membrane protein moiety and the interactive binding partner can be detected by measuring the amount of radioactivity that remains associated with the glutathione-agarose beads. A successful inhibition of the interaction by the test compound will result in a decrease in measured radioactivity.

Alternatively, the GST-membrane protein fusion protein and the interactive binding partner can be mixed together in liquid in the absence of the solid glutathione-agarose beads. The test compound can be added either during or after the species are allowed to interact. This mixture can then be added to the glutathione-agarose beads, and unbound material is washed away. Again the extent of inhibition of the interaction between the membrane protein moiety and the binding partner can be detected by adding a labeled antibody, and measuring the radioactivity associated with the beads.

In another embodiment of the invention, these same techniques can be employed using peptide fragments that correspond to the binding domains of a membrane protein and/or the interactive or binding partner (in cases where the binding partner is a protein), in place of one or both of the full length proteins. Any number of methods routinely practiced in the art can be used to identify and isolate the binding sites. These methods include, but are not limited to, mutagenesis of the gene encoding one of the proteins and screening for disruption of binding in a co-immunoprecipitation assay. Compensatory mutation(s) in the sequence encoding the second species in the complex can then be selected. Sequence analysis of the genes encoding the respective proteins will reveal the mutations that correspond to the region of the protein involved in interactive binding. Alternatively, one protein can be anchored to a solid surface, using methods described herein, and allowed to interact with and bind to its labeled binding partner, which has been treated with a proteolytic enzyme, such as trypsin. After washing, a relatively short, labeled peptide comprising the binding domain may remain associated with the solid material, which can be isolated and identified by amino acid sequencing. Also, once the gene coding for the intracellular binding partner is obtained, short gene segments can be engineered to express peptide fragments of the protein, which can then be tested for binding activity, and purified or synthesized.

For example, and not by way of limitation, a membrane protein moiety can be anchored to a solid material, as described herein, by making a GST-membrane protein fusion protein and allowing it to bind to glutathione-agarose beads. The interactive binding partner can be labeled with a radioactive isotope, such as ³⁵S, and cleaved with a proteolytic enzyme, such as trypsin. Cleavage products can then be added to the anchored GST-membrane protein fusion protein and allowed to bind. After washing away unbound peptides, labeled bound material, representing the intracellular binding partner binding domain, can be eluted, purified, and analyzed for amino acid sequence by well-known methods. Peptides so identified can be produced synthetically or fused to appropriate facilitative proteins using recombinant DNA technology.

### 7.6 PHARMACEUTICAL COMPOSITIONS

Compounds identified via assays such as those described herein may be useful, for example, in elaborating the biological functions of the described membrane proteins. Such compounds can be administered to a patient at therapeutically effective doses to treat any of a variety of physiological or mental disorders. A therapeutically effective dose refers to that amount of the compound sufficient to result in any delay in onset, or any amelioration, impediment, prevention, or alteration of any biological or overt symptom.

### 7.6.1 Effective Dose

Toxicity and therapeutic efficacy of such compounds can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index, and can be expressed as the ratio LD₅₀/ED₅₀. Compounds that exhibit large therapeutic indices are preferred. While compounds that exhibit toxic side effects may be used in certain embodiments, care should be taken to design a delivery system that targets such compounds to the site of affected tissue, in order to minimize potential damage to uninfected cells and, thereby, reduce side effects.

The data obtained from the cell culture assays and animal studies can be used in formulating a range of dosages for use in humans. The dosage of such compounds lies preferably within a range of circulating concentrations that include the ED₅₀ with little or no toxicity. The dosage may vary within this range depending upon the dosage form employed and the route of administration utilized. For any compound used in the methods of the invention, the therapeutically effective dose can be estimated initially from cell culture assays. A dose may be formulated in animal models to achieve a circulating plasma concentration range that includes the IC₅₀ (i.e., the concentration of the test compound that achieves a half-maximal inhibition of symptoms) as determined in cell culture. Such information can be used to more accurately determine useful doses in humans. Levels in plasma may be measured, for example, by high performance liquid chromatography.

When the therapeutic treatment of disease is contemplated, the appropriate dosage may also be determined using animal studies to determine the maximal tolerable dose, or MTD, of a bioactive agent per kilogram weight of the test subject. In general, at least one animal species tested is mammalian. Those skilled in the art regularly extrapolate doses for efficacy and avoiding toxicity to other species, including humans. Before human studies of efficacy are undertaken, Phase I clinical studies in normal subjects help establish safe doses.

Additionally, the bioactive agent may be complexed with a variety of well established compounds or structures that, for instance, enhance the stability of the bioactive agent or otherwise enhance its pharmacological properties (e.g., increase in vivo half-life, reduce toxicity, etc.).

The therapeutic agents will be administered by any number of methods known to those of ordinary skill in the art including, but not limited to, inhalation, subcutaneous, intravenous, intraperitoneal, intramuscular, or intrathecal injection, or topically applied (transderm, ointments, creams, salves, eye drops, and the like).

### 7.6.2 Formulation and Use

Pharmaceutical compositions for use in accordance with the present invention may be formulated in a conventional manner using one or more physiologically acceptable carriers or excipients. Thus, the compounds and their physiologically acceptable salts and solvates may be formulated for administration by inhalation or insufflation (either through the mouth or the nose) or oral, buccal, parenteral, intracranial, topical, intrathecal, or rectal administration.

For oral administration, the pharmaceutical compositions may take the form of, for example, tablets or capsules prepared by conventional means with pharmaceutically acceptable excipients such as binding agents (e.g., pregelatinised maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulphate). The tablets may be coated by methods well-known in the art. Liquid preparations for oral administration may take the form of, for example, solutions, syrups or suspensions, or they may be presented as a dry product for constitution with water or other suitable vehicle before use. Such liquid preparations may be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, ethyl alcohol or fractionated vegetable oils); and preservatives (e.g., methyl or propyl-p-hydroxybenzoates or sorbic acid). The preparations may also contain buffer salts, flavoring, coloring and sweetening agents as appropriate.

Preparations for oral administration may be suitably formulated to give controlled release of the active compound. For buccal administration the compositions may take the form of tablets or lozenges formulated in conventional manner.

For administration by inhalation, the compounds for use according to the present invention are conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of, e.g., gelatin for use in an inhaler or insufflator may be formulated containing a powder mix of the compound and a suitable powder base such as lactose or starch.

The compounds may be formulated for parenteral administration by injection, e.g., by bolus injection, or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. Alternatively, the active ingredient may be in powder form for constitution with a suitable vehicle, e.g., sterile pyrogen-free water, before use.

The compounds may also be formulated in rectal compositions such as suppositories or retention enemas, e.g., containing conventional suppository bases such as cocoa butter or other glycerides.

In addition to the formulations described previously, the compounds may also be formulated as a depot preparation. Such long acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

The compositions may, if desired, be presented in a pack or dispenser device that may contain one or more unit dosage forms containing the active ingredient. The pack may, for example, comprise metal or plastic foil, such as a blister pack. The pack or dispenser device may be accompanied by instructions for administration.

The present invention is not to be limited in scope by the specific embodiments described herein, which are intended as single illustrations of individual aspects of the invention, and functionally equivalent methods and components are within the scope of the invention. Indeed, various modifications of the invention, in addition to those shown and described herein, will become apparent to those skilled in the art from the foregoing description. Such modifications are intended to fall within the scope of the appended claims. All referenced publications, patents, and patent applications are herein incorporated by reference.

1. An isolated nucleic acid molecule that encodes the amino acid sequence of SEQ ID NO:2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 63, 65, 67, 69, 71, 73, 75, 77, 79, 81, 83, 85, 87, 89, 91, 93, 95, 97, 99, 101, 103, 105, 107, 109, 111, 113, 116, 119, 121, 124, 126, 128, 130, 133, 135, 137, 139, 142, 144, 146, 149, 151, 154, 157, 159, 162, 164, 166, 169, 172, 175, 177,180,182, 184, 186, 188, 191, 193, 196, 198, 201, 203, 206, 208, 210, 212, 214, 216, 218, 220, 223, 225, 227, 229, 233, 235, 237, 239, 241, 243, 245, 247, 249, 252, 254, or 257.

2. The isolated nucleic acid molecule of claim 1, wherein said nucleic acid molecule comprises the nucleotide sequence of SEQ ID NO:1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 62, 64, 66, 68, 70, 72, 74, 76, 78, 80, 82, 84, 86, 88, 90, 92, 94, 96, 98, 100, 102, 104, 106, 108, 110, 112, 115, 118, 120, 123, 125, 127, 129, 132, 134, 136, 138, 141, 143, 145, 148, 150, 153, 156, 158, 161, 163, 165, 168, 171, 174, 176, 179, 181, 183, 185, 187, 190, 192, 195, 197, 200, 202, 205, 207, 209, 211, 213, 215, 217, 219, 222, 224, 226, 228, 232, 234, 236, 238, 240, 242, 244, 246, 248, 251, 253, or 256.

3. An expression vector comprising the isolated nucleic acid molecule of claim 1.

4. (canceled)

5. An isolated polypeptide comprising the amino acid sequence of SEQ ID NO:2, 4, 6, 8, 10, 12, 14, 16, 18,20, 22, 24, 26, 28, 30, 32, 34, 36, 38, 40, 42 44, 46, 48, 50, 52, 54, 56, 58, 60, 63, 65, 67, 69, 71, 73, 75, 77, 79, 81, 83, 85, 87, 89, 91, 93, 95, 97, 99, 101,103, 105, 107, 109,111, 113,116, 119, 121, 124, 126, 128, 130, 133, 135, 137, 139, 142, 144, 146, 149, 151, 154, 157, 159, 162, 164, 166, 169, 172, 175, 177, 180, 182, 184, 186, 188, 191, 193, 196, 198, 201, 203, 206, 208, 210, 212, 214, 216, 218, 220, 223, 225, 227, 229, 233, 235, 237, 239, 241, 243, 245, 247, 249, 252, 254, or 257.

6. The isolated polypeptide of claim 5, comprising the amino acid sequence of SEQ ID NO: 2, 4, 6, 8, 10, 12, 14, 16, 18, 20, 22, 24, 26, 28, 30, 32, 34, 36, 38, 40, 42, 44, 46, 48, 50, 52, 54,56, 58, 60, 119, 121, 146, 149, 151, 157, 159, 196, 198, 201, 203, 223, 225, 227, 229, 233, 235, 237, 239, 241, 243, 245, 247, 249, 252, 254, or 257.

7. The isolated nucleic acid molecule of claim 1, wherein said nucleic acid molecule encodes the amino acid sequence of SEQ ID NO: 2, 4, 6, 8, 10, 12, 14, 16, 18, 20, 22, 24, 6, 8, 30 , 32, 34, 36, 38, 40, 42, 44, 46, 48, 50, 52, 54, 56, 58, 60, 119, 121, 146, 149, 151, 157, 159, 196, 198, 201, 203, 223, 225, 227, 229, 233, 235, 237, 239, 241, 243, 245, 247, 249, 252, 254, or 257.

8. The isolated nucleic acid molecule of claim 1, wherein said nucleic acid molecule comprises the nucleotide sequence of SEQ ID NO: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 118, 120, 145, 148, 150, 156, 158, 195, 197, 222, 224, 226, 228, 232, 234, 236, 238, 240, 242, 244, 246, 248, 251, 253, or 256.

9. An oligonucleotide that inhibits the expression of a nucleic acid molecule that encodes an amino acid sequence of SEQ ID NO: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 63, 65, 67, 69, 71, 73, 75, 77, 79, 81, 83, 85, 87, 89, 91, 93, 95, 97, 99, 101, 103, 105, 107, 109, 111, 113, 116, 119, 121, 124, 126, 128, 130, 133, 135, 137, 139, 142, 144, 146, 149, 151, 154, 157, 159, 162, 164, 166, 169, 172, 175, 177, 180, 182, 184, 186, 188, 191, 193, 196, 198, 201, 203, 206, 208, 210, 212, 214, 216, 218, 220, 223, 225, 227, 229, 233, 235, 237, 239, 241, 243, 245, 247, 249, 252, 254, or 257.

10. The oligonucleotide ofclaim 9, wherein said nucleic acid molecule encodes the amino acid sequence of SEQ ID NO: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 119, 121, 146, 149, 151, 157, 159, 196, 198, 201, 203, 223, 225, 227, 229, 233, 235, 237, 239, 241, 243, 245, 247, 249, 252, 254, or 257.

11. An antibody that selectively binds a polypeptide comprising an amino acid sequence of SEQ ID NO: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 63, 65, 67, 69, 71, 73, 75, 77, 79, 81, 83, 85, 87, 89, 91, 93, 95, 97, 99, 101, 103, 105, 107, 109, 111, 113, 116, 119, 121, 124, 126, 128, 130, 133, 135, 137, 139, 142, 144, 146, 149, 151, 154, 157, 159, 162, 164, 166, 169, 172, 175, 177, 180, 182, 184, 186, 188, 191, 193, 196, 198, 201, 203, 206, 208, 210, 212, 214, 216, 218, 220, 223, 225, 227, 229, 233, 235, 237, 239, 241, 243, 245, 247, 249, 252, 254, or 257.

12. The anitbody claim 11, wherein said polypeptide comprises the amino acid sequence of SEQ ID NO: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 119, 121, 146, 149, 151, 157, 159, 196, 198, 201, 203, 223, 225, 227, 229, 233, 235, 237, 239, 241, 243, 245, 247, 249, 252, 254, or 257.
